(19) United States
(12) Patent Application Publication
(10) Pub. No.: US 2009/0312689 A1
(43) Pub. Date: Dec. 17, 2009

(54) Adjustable Current Electrotransport Fentanyl Delivery Device

(75) Inventors: Amy Droitcour, San Francisco, CA (US); Janet A. Tamada, Stanford, CT (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: ALZA Corporation, Mountain View, CA (US)

(21) Appl. No.: 12/477,334

(22) Filed: Jun. 3, 2009

Related U.S. Application Data
(60) Provisional application No. 61/059,161, filed on Jun. 5, 2008.

(51) Int. Cl. A61N 1/30; H01S 4/00

(52) U.S. Cl. 604/20; 29/592.1

(57) ABSTRACT

A transdermal electrotransport fentanyl delivery system having relatively stable fentanyl flux. The system has a controller that controls a current through a donor reservoir wherein the current is generally changed nonlinearly with time with conductance or resistance as a factor to result in a relatively stable fentanyl flux.

### CROSS REFERENCE TO RELATED APPLICATIONS

This application claims the benefit of provisional application Ser. No. 61/059,161, filed Jun. 5, 2008, which is incorporated herein by reference in its entirety.

### TECHNICAL FIELD

The present invention relates to an electrotransport drug delivery system for driving fentanyl across a body surface or membrane. In particular, the invention relates to a system that delivers a current that generally changes with time to result in a relatively stable fentanyl flux.

### BACKGROUND

The delivery of active pharmaceutical agents through the skin provides many advantages, including comfort, convenience, and non-invasiveness. Gastrointestinal irritation and the variable rates of absorption and metabolism including first pass effect encountered in oral delivery are avoided. Transdermal delivery also provides a high degree of control over blood concentrations of any particular active agent.

In transdermal drug delivery, the natural barrier function of the body surface, such as skin, presents a challenge to delivery therapeutics into circulation. Devices have been invented to provide transdermal delivery of drugs. Transdermal drug delivery can generally be considered to belong to one of two groups: transport by a “passive” mechanism or by an “active” transport mechanism. In the former, such as DUROGESIC® fentanyl transdermal systems available from Janssen Pharmaceuticals and other drug delivery skin patches, the drug is incorporated in a solid matrix, or a reservoir with rate-controlling membrane, and/or an adhesive system.

Passive transdermal drug delivery offers many advantages, such as ease of use, little or no pain at use, disposability, good control of drug delivery and avoidance of hepatic first-pass metabolism. However, many active agents are not suitable for passive transdermal delivery because of their size, ionic charge characteristics, and hydrophilicity. Most passive transdermal delivery systems are not capable of delivering drugs under a specific profile, such as by ‘on-off’ mode, pulsatile mode, etc. Consequently, a number of alternatives have been proposed in which the flux of the drug(s) is driven by various forms of energy. Some examples include the use of iontophoresis, ultrasound, electroporation, heat and microneedles. These are considered to be “active” delivery systems.

One method for transdermal delivery of such active agents involves the use of electrical current to actively transport the active agent into the body through intact skin by electrotransport. Electrotransport techniques may include iontophoresis, electroosmosis, and electroporation. Electrotransport devices, such as iontophoretic devices are known in the art, see, e.g., EP0939659 A1 and U.S. Pat. Nos. 6,049,733, 6,181,963 and 6,216,033. One electrode, called the active or donor electrode, is the electrode from which the active agent is delivered into the body. The other electrode, called the counter or return electrode, serves to close the electrical circuit through the body. In conjunction with the patient's body tissue, e.g., skin, the circuit is completed by connection of the electrodes to a source of electrical energy, and usually to circuitry capable of controlling the current passing through the device. If the substance to be driven into the body is ionic and is positively charged, then the positive electrode (the anode) will be the active electrode and the negative electrode (the cathode) will serve as the counter electrode. If the ionic substance to be delivered is negatively charged, then the cathodic electrode will be the active electrode and the anodic electrode will be the counter electrode.

A prior iontophoretic system similar to that of U.S. Pat. No. 6,181,963 is shown in FIG. 1. FIG. 1 shows a perspective exploded view of an electrotransport device 10 having an activation switch in the form of a push button switch 12 and a display in the form of a light emitting diode (LED) 14. Device 10 includes an upper housing 16, a circuit board assembly 18, a lower housing 20, anodic electrode 22, cathodic electrode 24, anodic reservoir 26, cathodic reservoir 28 and skin-compatible adhesive 30. Upper housing 16 has lateral wings 15 that assist in holding device 10 on a patient's skin. Upper housing 16 is preferably composed of an injection moldable polymer.

Printed circuit board assembly 18 includes an integrated circuit 19 coupled to discrete electrical components 40 and battery 32. Printed circuit board assembly 18 is attached to housing 16 by posts (not shown) passing through openings 13a and 13b, the ends of the posts being heated/melted in order to heat weld the circuit board assembly 18 to the housing 16. Lower housing 20 is attached to the upper housing 16 by means of adhesive 30, the upper surface 34 of adhesive 30 being adhered to both lower housing 20 and upper housing 16 including the bottom surfaces of wings 15. Shown (partially) on the underside of printed circuit board assembly 18 is a battery 32, preferably a button cell battery and most preferably a lithium cell. Other types of batteries may also be employed to power device 10.

The circuit outputs (not shown in FIG. 1) of the circuit board assembly 18 make electrical contact with the electrodes 24 and 22 through openings 23,23′ in the depressions 25,25′ formed in lower housing, by means of electrically conductive adhesive strips 42,42′. Electrodes 22 and 24, in turn, are in direct mechanical and electrical contact with the top sides 44′, 44 of reservoirs 26 and 28. The bottom sides 46′, 46 of reservoirs 26,28 contact the patient's skin through the openings 29′, 29 in adhesive 30. The skin-facing side 36 of the adhesive 30 has adequate adhesive property to maintain the device on the skin for the duration of the use of the device.

Recently, there have been suggestions to control the current of drug delivery with more sophisticate current or voltage profiles. See, for example, U.S. Pat. No. 5,207,752, U.S. Pat. No. 5,983,130, U.S. Pat. No. 6,219,576, and EP941085B1. However, there has not been an electrotransport system that has been shown to deliver fentanyl with relatively stable flux over time.

What is needed is an electrotransport device that can deliver fentanyl with relatively stable flux over time.

### SUMMARY

The present invention relates to an electrotransport device for delivering fentanyl through the skin of a patient with a relatively stable fentanyl flux per dose. The present invention provides such electrotransport devices and methods of making and using such electrotransport devices in which the amount of electricity delivered through the system for drug delivery can be adjusted based on time and conductance.

In one aspect, the device has a donor compartment (e.g., reservoir) containing the fentanyl salt (a therapeutic agent for analgesic effect) for delivery through the body surface by electrotransport. The device has at least two electrodes for driving fentanyl ions from the donor reservoir to deliver a dose of fentanyl in a dose period. A sensor is present to sense a factor that is one of resistance (R) of the skin to current and conductance (C) of the skin to current. A controller is connected to the electrodes to control the current that drives the fentanyl ions such that the amount of charge per dose period passing through the donor reservoir varies as a function of time and as a function of the factor to result in a more stable fentanyl delivery than a constant charge per dose period delivery. In one aspect, the controller controls the drug delivery current such that the amount of charge per dose period passing through the donor reservoir decreases with time nonlinearly and nonexponentially and at a particular time, increasing the drug delivery current if a higher resistance (R) or a lower conductance (C) is sensed and decreasing the drug delivery current if a lower resistance (R) or a higher conductance (C) is sensed.

In one aspect, the controller controls the drug delivery current to have a time-varying base profile and adjusts the drug delivery current by increasing or decreasing the drug delivery current from the base profile according to the resistance (R) or conductance (C) sensed by the sensor.

In one aspect, the controller adjusts the drug delivery current to vary from the base profile with a function that is dependent on time and dependent on the resistance (R) or conductance (C) sensed.

It has been shown that if a constant current is used for electrotransport of fentanyl, the flux for the same dose period tends to be low initially and slowly increases with time. Since a more stable flux over time is desirable for controlling pain, we have shown that it is beneficial to start fentanyl delivery with a larger amount of current for the same dose period. Further, we have shown that the flux of the drug delivered at a particular current increases with conductance of the skin. Thus, with the present invention, a more stable flux with time is achieved by way of starting with a higher current and decreasing the current flow with time as well as adjusting the current based on the conductance of the skin. In this way, we were able to deliver fentanyl with an adequate flux initially and with more flux stability.

An electrotransport device that provides adequate drug administration in the initial period and throughout the time of use of the device provides significant advantage over devices that start out with low flux and increase the flux with time or without adjustment as a function of the conductance of the skin. With the devices of the present invention, adequate analgesia is possible from the beginning hour of the use of the device and drug flux is more stable.

The present invention also provides methods of making and methods of using the above electrotransport devices.

### BRIEF DESCRIPTION OF THE FIGURES

The present invention is illustrated by way of examples in embodiments and not limitation in the figures of the accompanying drawings in which like references indicate similar elements. The figures are not shown to scale unless indicated otherwise in the content.

FIG. 1 illustrates an exploded perspective view of a prior art typical electrotransport system.

FIG. 2 illustrates a schematic view of an embodiment of an electrotransport system of the present invention.

FIG. 3A is a schematic drawing illustrating the device having a sensor for sensing conductance or resistance of the skin and two reservoirs.

FIG. 3B is a schematic drawing illustrating the device having a sensor for sensing conductance or resistance of a reservoir.

FIG. 3C is a schematic drawing illustrating the device having a sensor for sensing conductance or resistance of the skin and a reservoir.

FIG. 4 is a graph showing data that illustrate fentanyl flux as a function of time and predicted fentanyl flux if the current is controlled according to an embodiment of base current profile of the present invention.

FIG. 5 is a plot of the fentanyl absorbed versus the conductance during the first hour for individuals in a 1-hour treatment.

FIG. 6 is a plot of the fentanyl absorbed versus the conductance during the first hour for individuals in a 3-hour treatment.

FIG. 7 is a plot of the fentanyl absorbed versus the conductance during the first hour for individuals in a 6-hour treatment.

FIG. 8 is a plot of the fentanyl absorbed versus the conductance during the first hour for individuals in a 9-hour treatment.

FIG. 9 is a plot of the fentanyl absorbed versus the conductance during the first hour for individuals in a 12-hour treatment.

FIG. 10 is a plot of the fentanyl absorbed versus the conductance during the second hour for individuals in a 3-hour treatment.

### DETAILED DESCRIPTION

The present invention is directed to an electrotransport drug delivery system that delivers fentanyl with an average amount of charge per dose period that has a general trend that falls with time to result in a more stable fentanyl flux over time. In particular, the system has a controller that controls the average amount of charge per dose period so that it decreases in a nonlinear and nonexponential way to drive fentanyl ions from the donor reservoir transdermally and further that the controller adjusts the current as a function of skin resistance or conductance. Through the transdermal delivery of fentanyl ions, active fentanyl moiety is delivered transdermally.

The practice of the present invention will employ, unless otherwise indicated, conventional methods used by those skilled in the art of mechanical and electrical connections in drug device development.

In describing the present invention, the following terminology will be used in accordance with the definitions set out below.

The singular forms “a,” “an” and “the” include plural referents unless the context clearly dictates otherwise. Thus, for example, reference to “a polymer” includes a single polymer as well as a mixture of two or more different polymers.

The term “AUC” means the area under the curve obtained in a subject by plotting blood plasma concentration of the beneficial agent in the subject against time, as measured from the time of start of dosing, to a time “t” after the start of dosing. AUC_{inf} is the area under the curve extended to time of infinity. For steady state, the AUCₙ is the area under the curve for a single dose normalized for doses administered. The AUC can be obtained by assaying samples from a patient.

As used herein, the term “Cₘₐₓ,” refers to the peak blood or plasma concentration of the drug, e.g., fentanyl.

As used herein, the term “Cₘᵢₙ” refers to the valley blood or plasma concentration of the drug, e.g., fentanyl.

As used herein, the term “bioavailability”, refers to the rate and extent to which the active ingredient or active moiety is absorbed from a drug product and becomes available at the site of action. The rate and extent are established by the pharmacokinetic-parameters, such as, the peak blood or plasma concentration (Cₘₐₓ) of the drug and the area under the blood or plasma drug concentration-time curve (AUC). To be bioequivalent to a prior transdermal drug delivery system, the 90% confidence interval of the steady state Cₘₐₓ ratio of a new transdermal system to that of the prior system needs to be within 80% to 125% and the 90% confidence interval of the steady state AUCₛₛ ratio of a new transdermal system to that of the prior system of the same dose strength needs to be within 80% to 125%. All delivery doses for fentanyl here are expressed in terms of fentanyl base equivalent unless specified otherwise in the content.

As used herein, “dose period” refers to a period of time during which the device delivers a nominal dose that the device has been designed to deliver. Such a nominal dose is typically a target amount of drug that the device is specified to deliver according to regulatory approval by a competent government drug administration agency. Typically such a dose is delivered each time the device is activated for delivery of a dose.

As used herein, the “trending down” of charge delivery (e.g., current) refers to a delivery profile in which during the delivery time the system is designed to be used, the averaged charge per dose of the second half of delivery is less than that of the first half of delivery.

### MODES OF CARRYING OUT THE INVENTION

The present invention provides an electrotransport device that is for electrotransport delivery of ionic fentanyl through a surface, such as skin.

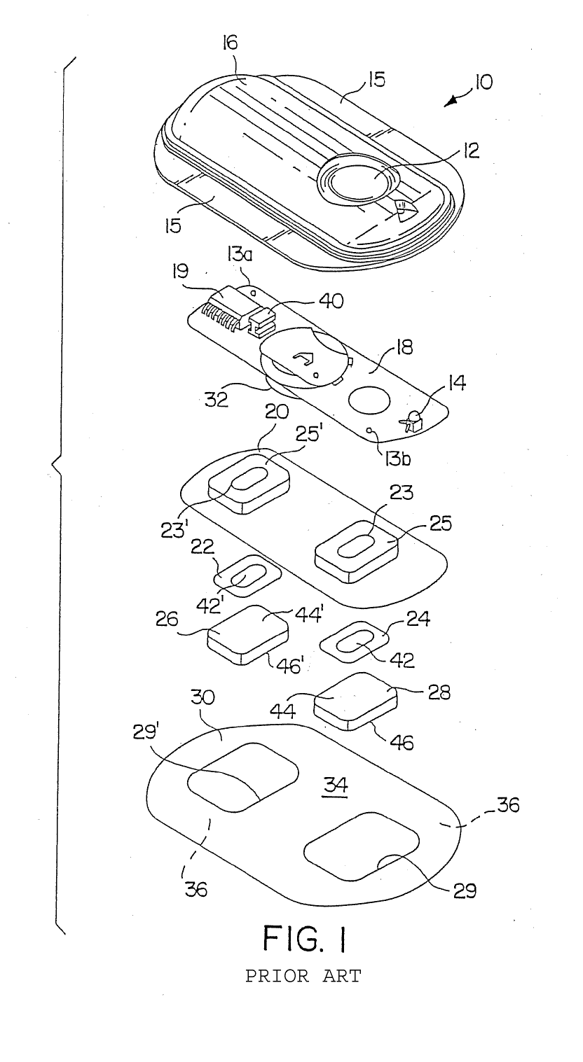

Electrotransport devices, such as iontophoretic devices are known in the art, e.g., U.S. Pat. No. 6,216,033. The structures, drugs, and electrical features of U.S. Pat. No. 6,216,033 and in FIG. 1 can be adapted to equivalents to be used in the present invention, as can be understood by one skilled in the art. In an iontophoretic drug delivery device, there is a drug reservoir and a counter reservoir.

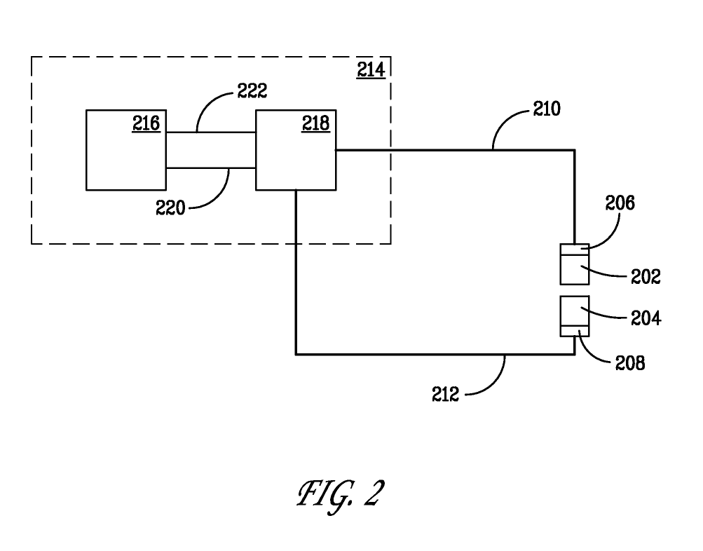

FIG. 2 shows a schematic representation of an embodiment of an electrotransport device of the present invention. The fentanyl electrotransport device 200 includes a drug reservoir 202 and a counter reservoir 204 connected by means of donor electrode (here the anode) 206 and counter or return electrode (here the cathode) 208 and conductors 210, 212 respectively to the electronic circuitry 214 of the device. The electronic circuitry 214 includes power source (e.g., battery) 216 and a controller 218 that controls the current flow through the electrodes 206, 208 during electrotransport. Conductors 220, 222 provide for electrical communication between the power source 216 and the controller 218. It is noted that FIG. 2 is just a schematic representation and one skilled in the art will know what detailed implementation can be used. For example, an embodiment can be made such that one of the electrodes can be at the electrical potential of the conductors 220, 222. On the other hand, an embodiment can be made that neither of the electrodes is at exactly the same electrical potential of the conductors 220, 222.

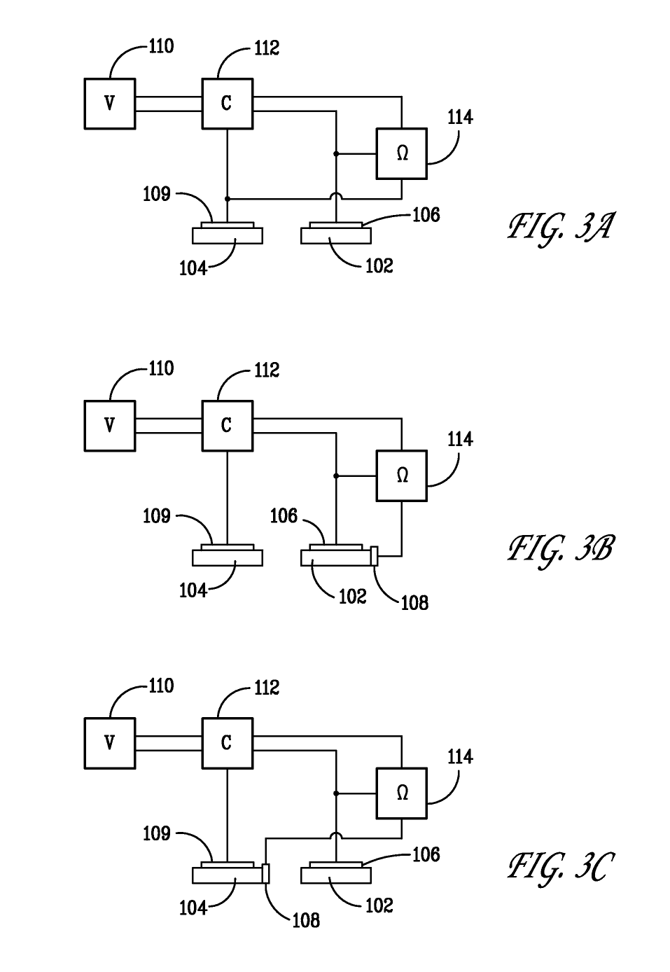

FIG. 3A is a schematic drawing illustrating the device having a sensor for sensing conductance of the skin. FIG. 3B shows a schematic representation of an embodiment of an electrotransport system having a resistance meter (ohmmeter) or conductance meter for determining the resistance (or to gauge the conductance) of a reservoir. In these figures, an ohmmeter is shown separate from the controller. If a resistance measurement were done as shown, it would be important that the controller have high input impedance so the resistance measurement is that of the electrodes, gels, and skin rather than the resistance in the controller. If switches were included such that the controller was disconnected during impedance measurement, this configuration could be used without concern about the controller impedance. In this figure the feature unrelated to the measurement of resistance is not shown so as to make the figure more easily understandable regarding resistance measurement. Again, the figures are schematic to illustrate a concept and the implementation can be done by one skilled in the art of circuit design. For the sake of clarity of description, reference below will generally be made on the measurement of resistance, although the measurement of conductance is applicable as understood by one skilled in the art. The electrotransport system 100 includes an ionic drug reservoir 102, a counter reservoir 104 that are to be placed on a body surface (not shown) for drug electrotransport. A donor electrode 106 contacts reservoir drug reservoir 102 to provide current for drug delivery. Counter electrode 109 contacts counter reservoir 104 for completing the electrical communication during electrotransport drug delivery to the body surface. Generally the donor electrode or a counter electrode is positioned centrally on a face of the corresponding reservoir so as to distribute current evenly over it. Voltage source 110 provides power for driving current flow. A Controller 112 that is operatively connected to the voltage source 110, donor electrode 106 and counter electrode 109 controls the operation of the electrotransport system, such as the duration of doses, turning the drug delivery system on or off based on various system conditions (e.g., out of range voltage or current flow), etc. A resistance meter 114 is connected to the donor electrode 106 and to the return electrode 109 to determine the resistance between the donor electrode 106 and the return electrode 109. In these schematic drawings, the skin on which the electrodes are applied is not shown for the sake of making the drawing less cluttered. The resistance meter 114 is in electrical communication to the controller 112 to provide resistance information. Resistance is typically sensed by sourcing a current through the device under test (DUT) and measuring the voltage required to source the known current, or, vice versa, to place a known voltage across the DUT and to sense the current. With a 4-wire measurement system, separate wires are used for sourcing the current and sensing the voltage. If a 4-wire measurement system was used with an electrotransport device, separate electrodes could be used within the same gel, or separate gels could be used that are located close to the drug delivery gels.

FIG. 3B provides a way that the resistance of a reservoir can be measured. The device contains an auxiliary electrode 108 that is connected to a face of the reservoir different from the face contacting the electrode through which the drug delivery current passes (e.g., the donor electrode 106) for the donor reservoir 102 and in contact with the skin. In this way, the resistance meter measures the resistance across the reservoir (e.g., donor reservoir 102) between the electrode (e.g., donor electrode 106) and the auxiliary electrode 108, providing information on the resistance of the reservoir. The auxiliary electrode 108 can be positioned to the side face of the donor reservoir as represented by FIG. 3B or on the side of another face of the donor reservoir 102 as long as it provides consistent measurement of the resistance of the reservoir. In this way, the auxiliary electrode (or monitoring electrode) is outside of the space between the donor electrode and the body surface. Similarly, connections can be configured to measure the resistance of the return reservoir. In this way, the resistance of the skin can be determined by subtracting the resistance of the reservoirs from the resistance across the donor electrode and return electrode.

FIG. 3C illustrates another embodiment in which the resistance between one reservoir and the skin can be sensed. Here, the resistance meter is connected to a drug delivery electrode (e.g., the donor electrode 106) and an auxiliary electrode 108 contacts the skin and contacts the reservoir that is in contact with the other drug delivery electrode (e.g., the counter electrode). The drug delivery electrodes are the electrodes through which the drug delivery current passes (i.e., the donor electrode and the counter electrode). A person skilled in art will understand that features that are applicable in the embodiment of FIG. 3A can similarly be applied in the embodiments of FIG. 3B and FIG. 3C.

It is noted that a resistance meter and a conductivity meter (or conductance meter) all amount to the same equivalent, in that the resistance or its inverse between two points are determined, i.e., whether it is determined in terms of resistance in ohms or conductance in Siemens (i.e., ohm⁻¹). Electrical resistance is a representation of how much an electrical component resists the flow of electrical current at a given voltage. Values of resistance and conductance can be determined and shown by devices such as ohmmeters. Further, since in certain embodiments of the present invention, the devices are designed to provide a known current, by sensing the applied voltage across two points (e.g., across the donor electrode and the return electrode), the resistance between the two points can be determined. Electronics internal to the device (e.g., with the programmable controller) can either use such information sensed, be it voltage, resistance, conductance, current, or other signals related thereto, to implement control to the effect that the current applied for drug delivery is dependent on the resistance (or conductance) of the skin as a factor.

It is noted that the resistance meter can be part of a body-surface-attaching unit, i.e., part of the portable electrotransport device that is attached to the body surface and carried around by the patient, or it can be a separate unit that is connectable and disconnectable to the portable electrotransport device. The circuits can be implemented on integrated circuit chips and installed either in a portable electrotransport device or placed in a separate unit that is connectable or disconnectable to the portable device. For example, if desired, either the portable electrotransport device or the resistance meter (or both) can have electrical receptors into which connectors from the other member of the electrotransport device/resistance meter pair can be physically inserted and frictionally fit or engage so that the connection can be frictionally maintained for resistance measurement without becoming disconnected. After the resistance measurement the connection is pulled apart on purpose to disengage the resistance meter. Such electrical receptors and connectors (e.g., prongs and sockets) are known in the art. Other connectors that can be used to engage the resistance meter with the portable electrotransport device can include clamps, clips, grips, and the like to provide disconnectable electrical communication. The designing of resistance measuring circuitry that can be implemented on a portable electrotransport unit is within the capability of one skilled in the art of such circuit design. It is preferable that the resistance meter be on the portable device itself.

The electrodes can be made with typical materials known in the art. For example, the anode electrode can be made with silver, the cathode electrode can be made with silver chloride, and if used, the auxiliary electrode(s) can be made with silver, silver chloride, nonconsumable material such as carbon, other metallic materials such as platinum, gold, titanium, tungsten, stainless steel, gold-plated material, etc., known to one skilled in the art.

In one embodiment the resistance (or conductance as its inverse) can be measured by sensing the voltage applied across two points and dividing by the current, which is either known or can be measured. This way of measuring resistance can be done during the time of regular drug delivery. In another embodiment, at a time when the drug delivery current is not flowing (e.g., between doses), a test current can be sent through the donor reservoir 102 to determine the resistance of the reservoir between two desired points, e.g., donor electrode 106 and the return electrode 104 or auxiliary electrode 108. The magnitude of the test current may be substantially less than what is necessary for driving therapeutic drug delivery, e.g., being less than 10% of the drug delivery current. The device can have a display or alert (e.g., light or sound or both) to alert the user that the resistance is outside a specific limit (e.g., a limit programmed, predetermined or preset) for that particular time. Impedance could also be measured using a pulsed or AC signal such that drug is not delivered but the skin impedance can still be measured.

Although it is possible to power the test current with the power source (battery) that drive therapeutic drug ions migration, the test current can be sent by the resistance meter from a power source that is different from the power source that drives the therapeutic drug ion migration.

It is noted that in the drug delivery system, the resistance meter and the controller can be separate units, or they can be an integral unit that can perform both functions. In fact, any of the integral unit, the controller, and the resistance meter can be an ASIC (application specific integrated circuit) or a design that incorporates programmable microprocessors, microcontrollers, or other discrete logic circuits and analog circuits. The designs of resistance measurement circuits, control circuits that can control voltage level and current level based on predetermined conditions such as changes in voltage, current, resistance, time, or other events are known to circuit designers skilled in the art. The resistance meter and the controller can be both present in the drug delivery system that is attachable to the body surface (“patch”). Alternatively, the resistance meter can be a separate unit that is physically connectable and disconnectable to plug into the controller unit for electrical communication.

The controller preferably controls the operation of the drug delivery system and directs the direction and magnitude of current flow through the various electrodes and their voltages such that the right levels of current and voltage are used for effective therapeutic ionic drug delivery by electrotransport, i.e., via a potential difference between the donor electrode and the counter electrode. Preferably the controller has circuitry that prevents a current flow to the body surface when the current or voltage during drug delivery is outside a predetermined range (based on safety reason) as can be determined by those skilled in the art. The controller can also control the drug delivery device to deliver the drug according to a regime, for example, dose, time interval, etc. Preferably, an advantageous feature of the controller is that it has the circuitry, either by programmable logic, or hardwired circuit, that can adjust the drug delivery current flow from the donor reservoir, through the body surface, such as that of the skin, to the counter reservoir. Preferably, the controller has circuitry that prevents the current flow to the body surface when the resistance across the donor reservoir or of the skin is outside a predetermined range.

For fentanyl, the controller 218 controls the dose of the drug delivered by transdermal electrotransport from about 20 μg (i.e., mcg) to about 60 μg (e.g., 40 μg of fentanyl) over a delivery time of up to about 20 minutes (delivery period or dose period, e.g., 10 min) in human patients having body weights of 35 kg or greater. Preferred is a dose size of about 30 μg to 60 μg of fentanyl per dose. Preferred is a dosage of about 35 μg to about 45 μg, and most preferred is a dosage of about 40 μg for the delivery period. The device of the invention further preferably includes means for delivering about 10 to 100, and more preferably about 20 to 80 additional like doses over a period of 24 hours in order to achieve and maintain the analgesic effect. When a user initiates (i.e., activates) the device, a dose is delivered. After the dose delivery is completed, if the device is initiated (or activated) again, another dose is delivered. The device can be made such that the controller cannot be activated for a second dose during the delivery of a first dose, and only a certain maximum number of doses can be activated per hour or per day, such as a maximum of six 10-minute doses per hour and 80 doses per day as in the IONSYS system. For example, the reservoir contains an amount of fentanyl salt (e.g., fentanyl halide, such as fentanyl hydrochloride) adequate for this dosing regime and the controller controls the delivery for such dosing practice. Other dose size, duration, and frequency of doses can be implemented by one skilled in the art.

The fentanyl salt-containing anodic reservoir formulation for transdermally delivering the above mentioned doses of fentanyl by electrotransport preferably contains an aqueous solution of a water soluble fentanyl salt such as halide salt (e.g., HBr or HCl) or citrate salts. Most preferably, the aqueous solution is contained within a hydrophilic polymer matrix such as a hydrogel matrix. The fentanyl salt is present in an amount sufficient to deliver the above-mentioned doses transdermally by electrotransport over a delivery period of preferably up to about 20 minutes, to achieve a systemic analgesic effect. The fentanyl salt typically contains about 1 to 10 wt % of the donor reservoir formulation (including the weight of the polymeric matrix) on a fully hydrated basis, and more preferably about 1 to 5 wt % of the donor reservoir formulation on a fully hydrated basis. Although not critical to the control aspect of the present invention, the applied electrotransport current density is typically in the range of about 50 to 150 μA/cm² (or mcA/cm²) and the applied electrotransport current is typically in the range of about 150 to 240 μA, depending on the analgesic effect desired. In the initial period of use of the device on a patient, the current can be slightly higher. The current can be up to about 380 μA. A smaller current can be used for a device of lower dose. For example, a device for 25 μg per dose can have a current of about 100 μA in the later part of delivery. In the initial periods of delivery, the current will correspondingly be slightly higher according to the present invention.

A suitable electrotransport device includes an anodic donor electrode, e.g., one that contains silver, and a cathodic counter electrode, e.g., one that contains silver chloride. The donor electrode is in electrical contact with the donor reservoir containing the aqueous solution of a fentanyl salt. The donor reservoir is preferably a hydrogel formulation. The counter reservoir also preferably comprises a hydrogel formulation containing a (e.g., aqueous) solution of a biocompatible electrolyte, such as citrate buffered saline. Typically the donor and the counter reservoir each would have a surface area for contacting skin of about 0.8 cm² to 10 cm². Preferably the anodic and cathodic hydrogel reservoirs preferably each have a skin contact area of about 1 cm² to 5 cm² and more preferably about 2 cm² to 3 cm². The anodic and cathodic hydrogel reservoirs preferably have a thickness of about 0.05 to 0.25 cm, and more preferably about 0.15 cm. Most preferably, the applied electrotransport current is substantially constant DC current during the dosing interval.

Preferably, the concentration of fentanyl in solution in the donor reservoir is maintained at or above the level at which the transdermal electrotransport fentanyl flux is independent of drug concentration in the donor reservoir during the electrotransport drug delivery period. Transdermal electrotransport fentanyl flux begins to become dependent upon the concentration of the fentanyl salt in aqueous solution as the fentanyl salt concentration falls below about 11 to 16 mM. The 11 to 16 mM concentration is calculated based only on the volume of liquid solvent used in the donor reservoir, not on the total volume of the reservoir. In other words, the 11 to 16 mM concentration does not include the volume of the reservoir that is represented by the reservoir matrix (e.g., hydrogel or other matrix) material. Furthermore, the 11 to 16 mM concentration is based upon the number of moles of fentanyl salt, not the equivalent number of moles of fentanyl free base, which is contained in the donor reservoir solution. For fentanyl HCl, the 11 to 16 mM concentration is equivalent to about 4 to 6 mg/ml. Other fentanyl salts (e.g., fentanyl citrate) will have slightly differing weight based concentration ranges based on the difference in the molecular weight of the counter ion of the particular fentanyl salt in question. As the fentanyl salt concentration falls to about 11 to 16 mM, the fentanyl transdermal electrotransport flux begins to significantly decline, even if the applied electrotransport current remains constant. Thus, to ensure a predictable fentanyl flux with a particular level of applied electrotransport current, the fentanyl salt concentration in the solution contained in the donor reservoir is preferably maintained above about 11 mM, and more preferably above about 16 mM.

Since fentanyl is a base, the salts of fentanyl are typically acid addition salts, e.g., citrate salts, hydrochloride salts, etc. The acid-addition-salts of fentanyl typically have water solubilities of about 25 to 30 mg/ml. When these salts are placed in solution (e.g., aqueous solution), the salts dissolve and form protonated fentanyl cations and counter (e.g., citrate, bromide or chloride) anions. As such, the fentanyl cations are delivered driven away from the anodic electrode of an electrotransport delivery device. In accordance with the teachings in these patents, the cationic fentanyl is preferably formulated as a halide salt (e.g., hydrochloride salt) so that any electrochemically-generated silver ions will react with the drug counter ions (i.e., halide ions) to form a substantially insoluble silver halide.

The reservoirs of the electrotransport delivery devices generally can contain a gel matrix, with the drug solution uniformly dispersed in at least one of the reservoirs. In IONSYS systems, the gel was made from poly(vinyl alcohol). Obviously, other types of reservoirs such as membrane-confined reservoirs are possible and contemplated. The application of the present invention is not limited by the type of reservoirs used. Gel reservoirs are described, e.g., in U.S. Pat. Nos. 6,039,977 and 6,181,963, which are incorporated by reference herein in their entireties. Suitable polymers for the gel matrix can contain essentially any synthetic and/or naturally occurring polymeric materials suitable for making gels. A polar nature is preferred when the active agent is polar and/or capable of ionization, so as to enhance agent solubility. Optionally, the gel matrix can be water swellable nonionic material.

Examples of suitable synthetic polymers include, but are not limited to, poly(acrylamide), poly(2-hydroxyethyl acrylate), poly(2-hydroxypropyl acrylate), poly(N-vinyl-2-pyrrolidone), poly(n-methylol acrylamide), poly(diacetone acrylamide), poly(2-hydroxylethyl methacrylate), poly(vinyl alcohol) and poly(allyl alcohol). Hydroxyl functional condensation polymers (i.e., polyesters, polycarbonates, polyurethanes) are also examples of suitable polar synthetic polymers. Polar naturally occurring polymers (or derivatives thereof) suitable for use as the gel matrix are exemplified by cellulose ethers, methyl cellulose ethers, cellulose and hydroxylated cellulose, methyl cellulose and hydroxylated methyl cellulose, gums such as guar, locust, karaya, xanthan, gelatin, and derivatives thereof. Ionic polymers can also be used for the matrix provided that the available counterions are either drug ions or other ions that are oppositely charged relative to the active agent.

The reservoir formulation for transdermally delivering cationic drugs by electrotransport is preferably composed of an aqueous solution of a water-soluble salt, such as HCl or citrate salts of a cationic drug, such as fentanyl. More preferably, the aqueous solution is contained within a hydrophilic polymer matrix such as a hydrogel matrix. The drug salt is preferably present in an amount sufficient to deliver an effective dose by electrotransport over a delivery period of up to about 20 minutes, to achieve a systemic effect. The drug salt typically includes about 0.05 to 20 wt % of the donor reservoir formulation (including the weight of the polymeric matrix) on a fully hydrated basis, and more preferably about 0.1 to 10 wt % of the donor reservoir formulation on a fully hydrated basis. In one embodiment the drug reservoir formulation includes at least 30 wt % water during transdermal delivery of the drug. Delivery of fentanyl by transdermal electrotransport of fentanyl cation has been described in U.S. Pat. No. 6,171,294, which is incorporated by reference herein. The parameter such as concentration, rate, current, etc. as described in U.S. Pat. No. 6,171,294 can be similarly employed here, since the electronics and reservoirs of the present invention can be made to be substantially similar to those in U.S. Pat. No. 6,171,294.

A preferred hydrophilic polymer matrix is polyvinyl alcohol such as a washed and fully hydrolyzed polyvinyl alcohol (PVOH), e.g. MOWIOL 66-100 commercially available from Hoechst Aktiengesellschaft. A suitable buffer is an ion exchange resin which is a copolymer of methacrylic acid and divinylbenzene in both an acid and salt form. One example of such a buffer is a mixture of POLACRILIN (the copolymer of methacrylic acid and divinyl benzene available from Rohm & Haas, Philadelphia, Pa.) and the potassium salt thereof. A mixture of the acid and potassium salt forms of POLACRLIN functions as a polymeric buffer to adjust the pH of the hydrogel to about pH 6. Use of a humectant in the hydrogel formulation is beneficial to inhibit the loss of moisture from the hydrogel. An example of a suitable humectant is guar gum. Thickeners are also beneficial in a hydrogel formulation. For example, a polyvinyl alcohol thickener such as hydroxypropyl methylcellulose (e.g. METHOCEL K100MP available from Dow Chemical, Midland, Mich.) aids in modifying the rheology of a hot polymer solution as it is dispensed into a mold or cavity. The hydroxypropyl methylcellulose increases in viscosity on cooling and significantly reduces the propensity of a cooled polymer solution to overfill the mold or cavity.

Polyvinyl alcohol hydrogels can be prepared, for example, as described in U.S. Pat. No. 6,039,977. The weight percentage of the polyvinyl alcohol used to prepare gel matrices for the reservoirs of the electrotransport delivery devices, in certain embodiments can be about 10% to about 30%, preferably about 15% to about 25%, and more preferably about 19%. Preferably, for ease of processing and application, the gel matrix has a viscosity of from about 1,000 to about 200,000 poise, preferably from about 5,000 to about 50,000 poise. In certain preferred embodiments, the drug-containing hydrogel formulation includes about 10 to 15 wt % polyvinyl alcohol, 0.1 to 0.4 wt % resin buffer, and about 1 to 30 wt %, preferably 1 to 2 wt % drug. The remainder is water and ingredients such as humectants, thickeners, etc. The polyvinyl alcohol (PVOH)-based hydrogel formulation is prepared by mixing all materials, including the drug, in a single vessel at elevated temperatures of about 90 degree C. to 95 degree C. for at least about 0.5 hour. The hot mix is then poured into foam molds and stored at freezing temperature of about −35 degree C. overnight to cross-link the PVOH. Upon warming to ambient temperature, a tough elastomeric gel is obtained suitable for ionic drug electrotransport.

Including fentanyl, a variety of drugs can also be delivered by electrotransport devices. In certain embodiments, the drug is a narcotic analgesic agent and is preferably selected from the group consisting of fentanyl and related molecules such as remifentanil, sufentanil, alfentanil, lofentanil, carfentanil, trefentanil as well as simple fentanyl derivatives such as alpha-methyl fentanyl, 3-methyl fentanyl and 4-methyl fentanyl, and other compounds presenting narcotic analgesic activity such as alphaprodine, anileridine, benzylmorphine, beta-promedol, bezitramide, buprenorphine, butorphanol, clonitazene, codeine, desomorphine, dextromoramide, dezocine, diampromide, dihydrocodeine, dihydrocodeinone enol acetate, dihydromorphine, dimenoxadol, dimeheptanol, dimethylthiambutene, dioxaphetyl butyrate, dipipanone, eptazocine, ethylmethylthiambutene, ethylmorphine, etonitazene, etorphine, hydrocodone, hydromorphone, hydroxypethidine, isomethadone, ketobemidone, levorphanol, meperidine, meptazinol, metazocine, methadone, methadyl acetate, metopon, morphine, heroin, myrophine, nalbuphine, nicomorphine, norlevorphanol, normorphine, norpipanone, oxycodone, oxymorphone, pentazocine, phenadoxone, phenazocine, phenoperidine, piminodine, piritramide, proheptazine, promedol, properidine, propiram, propoxyphene, and tilidine.

For more effective delivery by electrotransport salts of such analgesic agents are preferably included in the drug reservoir. Suitable salts of cationic drugs, such as narcotic analgesic agents, include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate. The more preferred salt is chloride.

A counterion is present in the drug reservoir in amounts necessary to neutralize the positive charge present on the cationic drug, e.g. narcotic analgesic agent, at the pH of the formulation. Excess of counterion (as the free acid or as a salt) can be added to the reservoir in order to control pH and to provide adequate buffering capacity. In one embodiment of the invention, the drug reservoir includes at least one buffer for controlling the pH in the drug reservoir. Suitable buffering systems are known in the art.

Some ionic drugs are polypeptides, proteins, hormones, or derivatives, analogs, mimics thereof. For example, insulin or mimics are ionic drugs that can be driven by electrical force in electrotransport.

### Method of Making

A device according to the present invention can be made by forming the various parts of the device (e.g., the parts as shown in FIG. 1) and assembling the parts into an assembled device. The polymeric layers such as the housing parts can be made by molding. Some of the layers can be applied together and secured. Some of the parts can be affixed together by adhesive bonding, interference fitting, or mechanical anchoring. Such chemical adhesive bonding methods, interference fitting and mechanical anchoring methods are known in the art. A device that delivers fentanyl with a more stable flux can be made as a single unit as shown in FIG. 1. Such a device is made by assembling the parts together to form a unit and then the assembled unit is packaged and stored until it is to be removed from the packaging for use. Alternatively, a device can be made by first making an electronic module and a reservoir module separately, wherein the two modules can be stored separately (e.g., in protected packages). The electronic module contains the control electronics and the reservoir module contains the reservoirs. The electronic module can be coupled with the reservoir module just before use by a medical professional, e.g., by inserting one module into the other module or by pressing the modules together. For example, the electronic module can be reusable whereas the reservoir module is disposable. After the system has been used, the reusable electronic module can be separated from the reservoir module and reused again by coupling with a fresh reservoir module. The used reservoir module can be discarded according to proper standard procedure. Iontophoretic drug delivery systems each with a separate drug reservoir unit and controller unit have been described in the past in patent and scientific literature. Such prior separate systems can be adopted for the electronic modules and reservoir modules of the present invention. For example, such assemble-before-use systems (electrotransport devices having parts being connected together before use) include those described in, U.S. Pat. No. 5,320,597 (Sage, Jr. et al); U.S. Pat. No. 4,731,926 (Sibalis); U.S. Pat. No. 5,358,483 (Sibalis); U.S. Pat. No. 5,135,479 (Sibalis et al.); UK Patent Publication GB2239803 (Devane et al), U.S. Pat. No. 5,919,155 (Lattin et al.), U.S. Pat. No. 5,445,609 (Lattin et al.); U.S. Pat. No. 5,603,693 (Frenkel et al.); and WO1996036394 (Lattin et al.).

Decreasing Amount of Charge Flow with Time

We have discovered that for a constant current delivery of fentanyl for a set length of time, the amount of fentanyl delivered changes depending on the time after the device has been put into use. The following pharmacokinetic evaluation demonstrated that the fentanyl flux is dependent on the time after the initial application. All fentanyl flux is determined in terms of flux of fentanyl base equivalent, which is well understood by those skilled in the art.

### Pharmacokinetic Evaluation

Fentanyl HCl patient-controlled transdermal analgesia (PCTA) system that delivers each requested dose over 10 minutes after initiation of dose was used. The 40 microgram (μg) PCTA systems were 40 μg dose IONSYS™ systems. The IONSYS™ system is structurally similar to that shown in FIG. 1 in general. IONSYS™ systems were designed to deliver 40 μg per dose with a duration of 10 minutes per dose with a maximum of 6 doses per hour for a maximum of 80 doses with a constant current of about 170 μA of current over a hydrogel reservoir of contacting surface area of about 2.7 to 2.8 cm². Each 25 μg system had a fentanyl HCl reservoir surface area of about 1.4 cm², with reduced current of about 100 μA.

Three studies were done, all 3 studies were single-center, open-label, randomized, and crossover, and the PCTA system was applied to the upper outer arm. Study I (25-μg system) evaluated 3 treatments: with different dosing frequencies: (1A) 2 sequential doses hourly for 23.33 hours, (1B) 6 sequential doses every 3 hours over 23.33 hours, and (1C) 72 sequential doses continuously. Study II (40-μg system) evaluated 3 treatments with different dosing frequencies: (2A) 2 sequential doses hourly for 23.33 hours; (2B) 6 sequential doses every 3 hours over 10 hours; and (2C) continuous sequential 80 doses. In Study III (40-μg system), subjects received 4 treatments (3A, 3B, 3C, and 3D): involving 6, 18, 36, and 80 continuous sequential doses over 1, 3, 6, and 13.33 hours, respectively. In treatment 3A only, two systems were used on a single individual simultaneously on two different locations on the skin. Thus a total of 12 doses (counting the two systems together) targeting 40 μg each were delivered in 1 hour to the individual in treatment 3A. Naltrexone was used to block fentanyl opioid effects. The mean weight of the individuals were 74.5 Kg, 72.6 Kg, and 70.4 Kg for Studies I, II, and III respectively. The nonparametric PK parameter area under concentration-time curve (AUC) was determined. Table 1 shows the treatment conditions.

TABLE 1
Fentanyl HCl Treatments Used Studies I, II, and III

| STUDY | TREATMENT | DESCRIPTION |
|---|---|---|
| I | 1A | 2 sequential doses/hour for 23.33 hours |
| | | (total doses = 48) |
| | 1B | 6 sequential doses every 3 hours 3 for 22 |
| | | hours (total doses = 48) |
| | 1C | 72 continuous doses over 12 hours |
| II | 2A | 2 sequential doses/hour for 23.33 hours |
| | | (total doses = 48) |
| | 2B | 6 sequential doses every 3 hours for 10 |
| | | hours (total doses = 24) |
| | 2C | 80 continuous doses over 13.33 hours |
| III | 3A | 2 systems each of 6 continuous doses over |
| | | 1 hour |
| | 3B | 18 continuous doses over 3 hours |
| | 3C | 36 continuous doses over 6 hours |
| | 3D | 80 continuous doses over 13.33 hours |

Table 2 shows AUCₜ (the AUC for the time period t) and how the AUCₙ (which is the AUC normalized per dose) values were estimated from AUCₜ.

TABLE 2

| TREATMENT | AUCₜ | AUCₙ (FOR SINGLE DOSE) |
|---|---|---|
| 1A | AUC₂₃₋₂₄ | AUC₂₃₋₂₄/2 |
| 1B | AUC₂₁₋₂₄ | AUC₂₁₋₂₄/6 |
| 1C | AUC_{inf} | AUC_{inf}/72 |
| 2A | AUC₂₃₋₂₄ | AUC₂₃₋₂₄/2 |
| 2B | AUC_{inf} | AUC_{inf}/24 |
| 2C | AUC_{inf} | AUC_{inf}/80 |
| 3A | AUC_{inf} | AUC_{inf}/12 |
| 3B | AUC_{inf} | AUC_{inf}/18 |
| 3C | AUC_{inf} | AUC_{inf}/36 |
| 3D | AUC_{inf} | AUC_{inf}/80 |

In Table 2, AUCₜ is defined in the AUCₜ column. AUC_{inf} is the AUC for the whole treatment. AUC₂₃₋₂₄ is the AUC after the 23^{rd} hour and throughout the 24^{th} hour. AUC₂₁₋₂₄ is the AUC after the 21^{st} hour up to and including the 24^{th} hour. The AUCₙ gives the estimate of how plasma fentanyl AUC might increase per dose of delivery for the dose that was delivered at the time corresponding to the period in AUCₜ. The AUC₂₁₋₂₄ and AUC₂₃₋₂₄ reflect the delivery rate at the end of the 24-hour period, whereas AUC_{inf} reflects the delivery rate averaged over the entire delivery period the system was worn.

Table 3 shows the pharmacokinetic data of Study I. Cₘₐₓ is the maximum serum fentanyl concentration as fentanyl base equivalent. Tₘₐₓ is the time at which Cₘₐₓ occurred. T_{1/2} is the half-life in hours. Ka is the elimination rate constant. The n numbers were the numbers of subjects with the Cₘₐₓ data. The numbers in the data are the mean values with the standard deviation in the parentheses.

TABLE 3
Pharmacokinetic Data of Study I

| | TREATMENT | | |
|---|---|---|---|
| | Fentanyl HCl | Fentanyl HCl | Fentanyl HCl |
| | PCTA 25 μg | PCTA 25 μg | PCTA 25 μg |
| PARAMETER | (1A) n = 25 | (1B) n = 25 | (1C) n = 25 |
| Cₘₐₓ, (μg/L) | 0.86 (0.30) | 0.97 (0.23) | 1.32 (0.31) |
| Tₘₐₓ, (h) | 23.11 (1.69) | 22.04 (0.19) | 10.56 (1.44) |
| t_{1/2}, (h) | — | — | 15.3 (12.0) |
| Ka (h⁻¹) | — | — | 0.067 (0.34) |
| AUCₜ (μg · h/L) | 0.75 (0.24) | 2.19 (0.54) | 22.58 (7.88) |
| AUCₙ (μg · h/L) | 0.374 | 0.365 | 0.314 |

Table 4 shows the pharmacokinetic data of Study II. It provides further data on AUC.

TABLE 4
Pharmacokinetic Data of Study II

| | TREATMENT | | |
|---|---|---|---|
| | Fentanyl HCl | Fentanyl HCl | Fentanyl HCl |
| | PCTA 40 μg | PCTA 40 μg | PCTA 40 μg |
| PARAMETER | (2A) n = 23 | (2B) n = 23 | (2C) n = 23 |
| Cₘₐₓ (μg/L) | 1.30 (0.30) | 0.91 (0.39) | 1.94 (0.43) |
| Tₘₐₓ (h) | 22.74 (1.46) | 10.07 (0.69) | 12.11 (1.24) |
| t_{1/2} (h) | 16.1 (5.5) | 15.1 (7.4) | 22.00 (10.0) |
| ka (h⁻¹) | 0.048 (0.015) | 0.061 (0.038) | 0.037 (0.014) |
| AUCₜ (μg · h/L) | 1.14 (0.26) | 10.3 (3.8) | 40.8 (12.7) |
| AUCₙ (μg · h/L) | 0.57 | 0.43 | 0.51 |

Table 5 shows the pharmacokinetic data of Study III.

TABLE 5
Pharmacokinetic Data of Study III

| | TREATMENT | | | |
|---|---|---|---|---|
| | Fentanyl HCl | Fentanyl HCl | Fentanyl HCl | Fentanyl HCl |
| | PCTA 40 μg | PCTA 40 μg | PCTA 40 μg | PCTA 40 μg |
| PARAMETER | (3A) n = 20 | (3B) n = 20 | (3C) n = 20 | (3D) n = 20 |
| Cₘₐₓ (μg/L) | 0.27 (0.18) | 0.72 (0.20) | 1.08 (0.48) | 2.00 (1.24) |
| Tₘₐₓ (h) | 1.86 (1.79) | 3.45 (0.33) | 6.12 (0.68) | 13.00 (1.93) |
| t_{1/2} (h) | 19.2 (6.0) | 10.5 (2.5) | 10.4 (4.6) | 20.5 (7.2) |
| ka (h⁻¹) | 0.040 (0.013) | 0.070 (0.016) | 0.076 (0.025) | 0.038 (0.015) |
| AUC_{inf} (μg · h/L) | 2.79 (1.43) | 5.75 (2.13) | 12.87 (5.32) | 39.88 (18.34) |
| AUCₙ (μg · h/L) | 0.23 | 0.32 | 0.36 | 0.50 |

From Table 5, it can be clearly seen that the mean dose amount increases as the duration of the study increases (as shown by the increase in value of AUCₙ). The data in Table 5 can be analyzed to determine the mean dosing at different dose intervals. Subtracting the AUC_{inf} for treatment 3A (0 to 1 hour) divided by 2 (to account for 2 systems applied in that study) from treatment 3B (0 to 3 hours) gives the AUC_{inf} for the 1 to 3 hour interval. Dividing that by the number of doses (12 in this case), gives the AUCₙ over the 1 to 3 hour interval. Similarly, the AUCₙ over the 3 to 6 and 6 to 13.33 hour intervals can be determined. The ratio of the dose for each interval compared to an estimate of the dose at 24 hours (estimated from data from a separate cohort of subjects at 23 to 23.33 hours) is given in the following table (Table 5A).

TABLE 5A

| | Flux Ratio |
|---|---|
| Time interval | (Flux in interval compared to flux |
| (hours) | at 23 to 23.33 hours) |
| 0 to 1 | 0.41 |
| 1 to 3 | 0.64 |
| 3 to 6 | 0.69 |
| 6 to 13.33 | 0.97 |

The results of the Studies I, II, and III indicated the fentanyl amount absorbed from the PCTA system increases as a function of time and is independent of dosing frequency and total number of doses delivered. The AUCₙ values from Study III were used to derive a mathematical model as a function of time. Data from the 4 points at t=0.5, 2, 4.5, and 9.66 hours (the midpoint of each measuring interval, i.e., 0 to 1 hour, 1 to 3 hour, 3 to 6 hour, and 6 to 13.33 hour) of Study III were used to construct a graph of delivery vs. time. Values were fitted to a curve and the best-fit curve was found to be nonlinear and nonexponential. Modeling the data with a second order polynomial fit lead to the following Equation (1). The per-dose flux, y(t), of fentanyl delivered expressed as a dimensionless decimal fraction of the targeted dose size or nominal dose size (e.g., 40 μg in a 40 μg per dose device) is the following Equation (1), wherein t is time in hours after initial activation of the system.

For t<12 hours,

y(t)=0.4027+0.0873t−0.0031t² (1)

Thus, the experimental data show that actual fentanyl flux tends to decrease with time for the same current flow in the same system; y(t) is an indication of how effective the current is producing flux of the drug.

At a constant current, the flux F(t) as related to the nominal dose flux Fo (e.g., 40 μg per 10 min in a 40 μg per 10-min dose device) is:

F(t)=Fo·y(t) (2)

Decreasing Current as with Time for More Consistent Flux

Since the experimental data of Studies I, II, and III show that actual fentanyl flux tends to decrease with time for the same current flow (between the donor and counter electrodes), to provide an actual flux that is more consistent or stable over time, it is preferred that the current used for driving drug flux is adjusted with time to have a downward trend. In other words, it is preferred that as a whole, the current at the later part of the use of the device be smaller (delivering less charge per unit time) than the current at the start. Based on Equation (1), it was found that the average current for the first hour of use is preferably between 1.7 and 2.7 times, more preferably 1.8 to 2.3 times, the average of the current to be applied at the final hour of designed use (e.g., hour 24 (i.e., after the 23^{rd} hour before the end of the 24^{th} hour) for a device designed for 24 hour use) if the device is projected to deliver current for a particular period (e.g., for 24 hours). The current would trend down (i.e., be adjusted downward) in general in a particular manner, even if the device (e.g., a 24 hour device) is taken off the skin before the end of the 24-hour period, e.g., because patient needed an MRI (magnetic resonance Image). If I_{f} is assumed to be the average current for a steady state delivery of the targeted dose (in most cases, e.g., for applications more than 12 hours, the final hour of use can be assumed to be the steady state), the current at time t that is needed to result in a stable flux (i.e., a per-dose flux in decimal fraction equal 1) is I(t):

I(t)=I_{f}/y(t) (3)

Thus, Equation (3) is a profile for the drug delivery current to follow to achieve a more stable flux over time. In Equation (3), y(t) is y(t)=0.4027+0.0873t−0.0031t² as stated above in Equation (1). In Equation (3), I(t) is related to the average amount of charge passing through the donor reservoir during a dosing period at time t and I_{f} is related to the average amount of charge passing through the donor reservoir during a dosing period at the final period of use. Because the sizes of the reservoirs in the device are constant, Equation (3) can also apply to the current density such that I(t) and I_{f} are current densities.

As an example, if a device is designed for 24 hour use with a constant dose period (e.g., 10 min) throughout the use of the device, and the target dose is 40 μg/dose, the average current at the 24^{th} hour is taken to be I_{f}. The fentanyl flux in decimal fraction of the target dose of 40 μg is y(t) according to Equation (1) at any time t. Thus, y(t) is a representation of the effectiveness of the current delivery in resulting in fentanyl flux as compared to the steady state (approximate by the final period of delivery) in the use of the device. Experimental data have shown that after about 10 hours of use, the effectiveness does not change substantially and can be assumed to be close to about 100%. At about 24 hours, the effectiveness can be assumed to be 100%.

To test for the current delivery profile with time, the device can be activated at maximum use for a period to test for the current and the amount of charge delivered. The term “at maximum use” means that the device is activated to deliver as many doses as the device is designed to deliver during a specific period. For example, if a device is designed to deliver 6 doses per hour, then at maximum use, no matter how often the device is activated during the period of one hour, the device delivers a maximum of 6 doses per hour. As long as the device can be operated at maximum use for time periods (e.g., for 1 hour, 2 hours, 3 hours, etc.) for the average flux per hour to be determined at various time periods during use, it is not necessary that the device be in fact in operation at maximum use for the whole period of time the device is in use.

To show whether a device behaves similar to Equation (1) and Equation (3), an average current passing through the donor reservoir over a period of time (e.g., one hour, 2 hour, 3 hour, etc.) can be estimated by obtaining the current-time graph under maximum use and dividing the area under the curve of the current time graph by time over the period. Similarly, the average amount of charge passing through per dosing period over a period (e.g., one hour, 2 hour, 3 hour, etc.) can be estimated by obtaining a current-time graph for each dose, summing up the total area under the curves for the doses and dividing by the number of doses during the period. Preferably, the estimations are done for a device under maximum use to obtain information about the characteristics of the device.

It is noted after a device has been used over the length of time it is designed to operate, the device is removed. If analgesia is still needed and a fresh device is to be applied to the skin, the fresh device is applied to a new location. Since the skin at the new location is not accustomed to fentanyl delivery by the fresh device, the current profile needs to be implemented all over again on the fresh device at the new location on the skin.

According to an embodiment of the present invention, in the downward trend of current delivery, a useful device wherein the fentanyl flux through human skin is relatively stable, the average amount of charge per dose of fentanyl flux differs from the average amount of charge per dose in an immediately subsequent hourly period by not more than 40%, preferably not more than 30%, more preferably by not more than 25% of that of the earlier hourly period. In an embodiment designed for at least 12 hours of use, preferably designed for 1 day of use, or for more than one day of use, in which the duration (per dose) of the dosing current flow remains unchanged over time but the current is changed, the average charge flow per dose period averaged over an earlier hourly period preferably differs from the average charge flow per dose period of an immediately subsequent hourly period by not more than 40%, more preferably not more than 30% of that of the earlier hourly period. It is also preferred that the average current (or the average charge flow per dose period) at the last hour (e.g., the 24^{th} hour for a 24 hour device) of designed use differs from the average current (or the average charge flow per dose period) of the initial hour of use by not more than 60%, preferably not more than 55%, more preferably not more than 50% of the average current of the initial hour of use. It is also preferred that the average current (or the average charge flow per dose period) at the last hour of designed use differs from the average current (or the average charge flow per dose period) of the initial hour of use by not less than 25%, preferably not less than 40% of the average current of the initial hour of use. It is also preferred that the average current (or the average charge flow per dose period) from the fourth hour through the sixth hour (i.e., after the third hour, through the sixth hour averaged over 3 total hours) of use differs from the average current (or the average charge flow per dose period) of the initial hour by not more than 50%, preferably not more than 40% of the average current of the initial hour (i.e., the first hour). It is also preferred that the average current (or the average charge flow per dose period) from the seventh hour through the ninth hour of use differs from the average current (or the average charge flow per dose period) from the tenth hour through the twelfth hour of use by not more than 30%, preferably not more than 20% of the average current of the seventh hour through the ninth hour of use. It is also preferred that the average current (or the average charge flow per dose period) from the seventh hour through the ninth hour of use differs from the average current (or the average charge flow per dose period) of the last hour of use by not more than 30%, preferably not more than 20% of the average current of the seventh hour through the ninth hour of use. It is also preferred that the average current (or the average charge flow per dose period) from the tenth hour through the twelfth hour of use differs from the average current (or the average charge flow per dose period) of the last hour of use by not more than 25%, preferably not more than 15% of the average current of the tenth hour through the twelfth hour of use. It is noted that it is not necessary to have the electrotransport current continuously decreasing with time. If desired, the current for a period of time can remain unchanged, or even go up in amplitude so long as the average current trends down as described in the above. Also, it is to be understood that when it is mentioned that the current having a particular profile with time with a dose period that remains constant, the invention can be practiced by providing the corresponding coulombs of charge per dose of drug via changing the duration of dose period and/or the current amplitude.

It is possible that during the period of use of the device on the skin, in any given period of time the user would refrain from activating the device or choose to activate only sparingly (e.g., during the first hour of use) and thus rendering the average current use per hour in reality to be zero or near zero during this period. However, the device is made in such a way that if it is activated for maximum use during a period (e.g., one hour or more) it can deliver current (or amount of charge passing through per dose period) that trends down as described above to provide stable fentanyl flux. It is not necessary that the device in fact be activated for maximum use or if activated for maximum use that it does so all the time during which the device is in use.

Although the charge delivery (e.g., current or duration or both) of the transdermal electrotransport device can be controlled manually, it is preferable that the control is electronic by a controller to provide the time-varied current. First, in certain embodiments, the charge delivery (e.g., current) is controlled to deliver in discrete periods of constant current for fentanyl delivery for at least a portion of the whole period or episode of use. Thus, for a period of time, the current is held constant until after the end of that period the current is changed to a different amplitude for another set period of time, etc. For Example, the current is held at a constant current of A μA for one hour and then immediately is switched to B μA for a period of two hours, before being switched to a constant current of C μA for another period of 3 hours thereafter. Alternatively, the current can be controlled, typically electronically, e.g., by means of hard wired circuitry, application-specific integrated circuit (ASIC), programmable circuitry (such as microprocessor(s)), microcomputer, etc., to continuously change the current with time. Such implementation of circuitry for control of current delivery is known to those skilled in the art of electrical control of medical devices. As used herein, if the device has discrete steps of current amplitude change and the number of stepping periods of different current amplitude exceeds 50 for the designed length of delivery, it is considered to be changing the current substantially continuously. If the number of stepping periods exceeds 100, the current can be considered to change continuously. Techniques and electronics on programming current control in medical devices are known to those skilled in the art. Thus, such skilled persons will be able to design and implement current control, either in discrete steps or with continuous change for electrotransport based on the present disclosure. For example, the “stepped-down” current control required to achieve a more stable fentanyl delivery (to approach a zero-order profile, zero-order meaning that the amount of fentanyl delivered per dose remains constant with time) can be accomplished with a programmable 8-bit microprocessor, the simplest computer architecture found in commercial applications. Examples of 8-bit microprocessors include the INTEL™ 8051 family, and the MOTOROLA™ 6800 family, which are familiar to those skilled in the art of programming control of drug delivery devices. Further, although the above examples are selected as programmable microcontrollers to provide both the required product functions and flexibility for implementing a multigenerational iontophoretic fentanyl delivery product line, there are other means that could provide the current control required for zero-order fentanyl delivery. Some examples of such other means include an uncommitted logic array (ULA), a field programmable gate array (FPGA), or even an analog circuit.

With the current controlled to deliver more current during the first part of the fentanyl delivery than in the later part to overcome the inefficiency of fentanyl delivery initially (i.e., regarding flux per current use), the current density as a consequence also decreases with time. However, even with increased current initially, it is preferred that the device is controlled to deliver the current at an average current density of less than 400 μA/cm² averaged over an hour, during the whole period of use for a 40 μg per 10 minute device. For a device that delivers a different dose strength dose, e.g., 25 μg per 10 minutes, 50 μg per 10 minutes, or 60 μg per 10 minutes, etc., the current density can be proportionally scaled to increase the charge delivery. More preferably, the averaged current density for any one hour is controlled to be less than 200 μA/cm², preferably less than 150 μA/cm² averaged over an hour at all time of use.

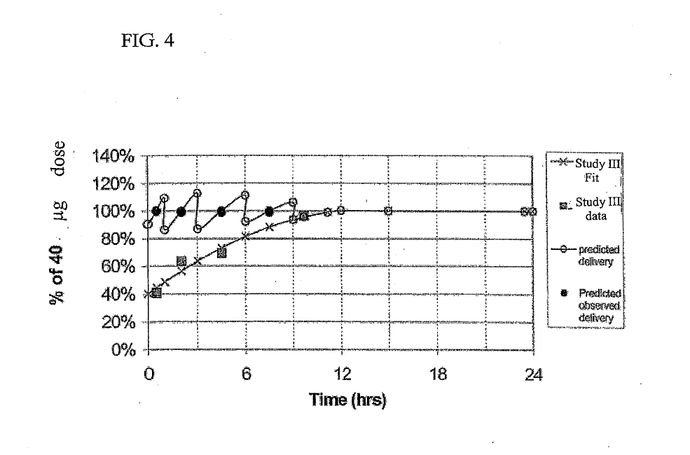

FIG. 4 is a graph showing data that illustrates fentanyl flux in human trial Study III, described above as a function of time and shows predicted fentanyl flux if the current is controlled according to an embodiment of the present invention. The ordinate of the graph shows the fentanyl flux as a percentage of target flux (e.g., a target flux of a 40 μg device is 40 μg/(10 min) of fentanyl). In FIG. 4, the squares are data points derived from Study III above. The smooth curve with “x” data points is the modeled line corresponding to Equation (1). The open circles are the predicted flux at the time indicated in the graph with current that trended downward in amplitude stepwise, the steps occurring at the end of the 1st hour, 3rd hour, 6^{th}, and 9^{th} hour. The flux is predicted by Equation (1), multiplying the flux by the ratio of the current at the time interval to the current at 24 hours. The solid disks represent the average predicted flux that would be observed at the mid point of each period of stepwise current change. The flux was calculated by using a trapezoidal approximation to determine the areas underneath the graph and dividing by time.

The following modeling method was used. A prediction based on the available clinical data from delivery rates for a 5-step current profile was made. This prediction was made in the following way:
Data from the 4 points at t=0.5, 2, 4.5, and 9.66 hours (the midpoint of each measuring interval) was used to construct a graph of delivery vs. time. Values were fitted to a second order polynomial fit of the data. The equation (Equation (1) curve-fitting to the data of Study III) was: y=0.4027+0.0873t−0.0031t², where t is time in hours and y is a unitless ratio, i.e., the fraction of the 40 μg dose delivered during the 10 minutes dose period. Time points after 12 hours were assumed to be at 100% delivery rate (40 μg).
Drug delivery was assumed to be directly proportional to current; i.e., a linear relationship with an intercept of zero was assumed for the relationship between dose delivered and current. Current step intervals of 0 to 1 (i.e., 1 hour through Hour 1 before Hour 2), 1 to 3 (i.e., 2 hours starting after Hour 1 through Hour 3), 3 to 6, and 6 to 9, and 9 to 24 were used. These were selected from the observation that change occurred more rapidly immediately after system activation than later in the application period. The intervals are therefore 1, 2, 3, 3, and 15 hours long.
The current level was determined by locating the percent delivered in the original IONSYS™ at the mid point of each interval (i.e., 1, 3, 6, and 9 hours) and dividing that into 170 μA, which is the current applied by the IONSYS™ system. The current to be applied is estimated by the equation I=I_{f}/y, wherein I_{f} is current where the value of the ratio y is estimated to be 1, which we have found to be generally about the average current for a targeted final hour of use (for a 24 hour system, it is the final hour of the day, i.e., time from Hour 23 to Hour 24, i.e., end of Hour 23 through Hour 24). The time variable, t, is time in hour after initial application on the skin. For example, from the graph, at 1 hour, delivery was 44% of the 40 μg dose. To increase the flux at the first hour to a level about equal to the desired flux (the desired flux is about equal to flux at the final hour), the current chosen for the 0 to 1 hour interval was 170 μA/0.44=382 μA.

Thus, the model predicts the current use to result in a fentanyl flux that is more stable. The following Table 6 shows the current levels and stepped changes used in the simulation.

TABLE 6

| | Deliver % | | current |
|---|---|---|---|
| | midpoint | | density |
| Time (hrs) | from fit | current (μA) | (μA/cm²) |
| 0 to 1 | 44% | 383 | 136 |
| 1 to 3 | 56% | 301 | 108 |
| 3 to 6 | 73% | 232 | 83 |
| 6 to 9 | 88% | 193 | 69 |
| 9 to 24 | 100% | 170 | 61 |

The above Table 6 is an example of a current profile to be used as a base profile on which the current profile can be modified based on resistance changes. Other current profiles can also be used as the base profile. For example, data from applying a current profile such as that in Table 6 can further be modeled to obtain another base current profile on which adjustment can be made based on resistance changes. Another example is simply a constant current base profile.

### A Stepwise Decrease of Electrotransport Current Profile

The following is an exemplary illustration of how a current profile is obtained on which the current can be fine-tuned based on resistance changes. The system used in this study includes a clinical controller (which includes microcontroller that was programmed to direct the delivery of a stepped-down current profile as well as a boost converter with feedback to operate as a current source that can be controlled by the microcontroller). The drug unit controlled by the controller included a fentanyl reservoir and a counter ion reservoir similar to the IONSYS system. The fentanyl reservoir hydrogel had an area of about 2.7 to 2.8 cm². The controller in this system was controlled by a programmable microprocessor and delivered the stepped-down current profile, ranging from a 380 μA maximum to a 170 μA minimum. The currents and intervals are shown in the table below. This profile was designed to target near-zero-order absorption of a 40 μg fentanyl dose over a 24-hour period. The controller (Model 1508-BD, a custom programmable current controller and includes a Microchip 16F84 8-bit microprocessor, and a Linear Technologies LT1109 boost converter) had a pre-programmed current profile that set the current during the clinical study. The drug unit was a minor modification of an existing design of an electrotransport (fentanyl HCl) 40 μg System (IONSYS™ System). The fentanyl reservoir hydrogel at the anode had an area of about 2.8 cm². The composition of the anode and cathode (counterion) hydrogels was identical to that of IONSYS™. The housing for the drug unit had conductive snaps to make electrical contact with the controller. Effectively, the reservoirs, except being connected to a controller that controlled the current to be stepped down with time, had the same structure and ingredient, and electrical connection as the IONSYS™ system.

This study included 40 healthy adult subjects, 34 of which completed an IV treatment of fentanyl and all 4 electrotransport treatments of fentanyl: a 1-hour treatment, a 3-hour treatment, a 6-hour treatment, and either a 9-hour treatment or a 12-hour treatment. During each treatment the serum fentanyl was measured periodically so the amount of fentanyl absorbed by each subject could be calculated. The desired delivery of fentanyl is 240 μg/hour, which is equivalent to six 10-minute 40 μg doses, with 60 minutes per hour. During the treatments, the controller recorded battery voltage, output voltage, and load current once per minute.

The modeled % efficiency at mid period is the estimated % effectiveness by the system to achieve the desired flux for the current delivered at the mid period of a period according to the model of Equation (1). In order to achieve a stable fentanyl flux with time with the estimated % effectiveness, the current applied was increased at the first part of the delivery according to Equation (3) as compared to the later part of the delivery. Table 7 shows the applied current and the resulting current density, as well as the resulting measured average fentanyl flux (in μg/hr) data for each of the periods of the stepped current. The result indicated the fentanyl flux was more stable with time than the result of fentanyl delivery without stepped down current as observed in Study III (see Tables 2 to 5 and FIG. 4 for comparison on systems without such stepped current profile).

TABLE 7
Data of Human Trail PK1

| | | | | | Flux as |
|---|---|---|---|---|---|
| | Modeled % | | Current | Measured | ratio to |
| Time | efficiency at | Current | density | averaged | 40 μg per |
| elapsed, hr | mid period | applied, μA | μA/cm² | flux, μg/hr | 10 min |
| 0-1 | 44 | 380 | 136 | 299 | 1.25 |
| 1-3 | 56 | 300 | 108 | 250 | 1.05 |
| 3-6 | 73 | 230 | 83 | 175 | 0.73 |
| 6-9 | 88 | 190 | 69 | 230 | 0.96 |
| 9-12 | 100 | 170 | 61 | 198 | 0.82 |

It is noted that with the application of current according to Table 7 (which rounded the numbers to approach the target currents of Table 6), the fentanyl flux during the first periods, i.e., for the 0-1 and 1-3 hour periods, the flux was slightly higher than the later periods. The data collected on conductance showed that the skin conductance generally fell with time, showing that ion permeability increased with time. Although the application of the present invention does not depend on any particular scientific theory, it is estimated that the effectiveness of current flow to result in fentanyl flux is affected by time. In Table 6, while the applied current was controlled to fall stepwise with time, the flux fell first, but at the 6-9 hours increased a little before falling at the 9-12 hours. Thus, it is estimated that to provide an even more stable fentanyl flux with time, the initial current to be used would be slightly less initially and slightly more during the 3-6 hour period part of the use of the device compared to the delivery directed by Equation (1) and Equation (3). Preferably, the base current profile used trends down with time up to and through about 6 hours. Then the current applied can stabilize or rise slightly before trending down again. Therefore, preferably, the difference between the initial and later part of the use of a device in current, as well as in flux is less pronounced that those shown in Table 7. Such modified current profiles can be precalculated by modeling normalized flux (e.g., relative to dose of 40 μg per 10 min) as various dose periods to arrive at close to a value of 1. This would result in a more stable flux, preferably for a reservoir of about 2 cm² to 3 cm², even more preferably about 2.7 to 2.8 cm².

Further, other than controlling the current so that the current generally trends down with time to result in a more stable fentanyl flux with time, it is understood that the device can be controlled so that generally the rate of charge passing through the donor reservoir per dose generally trends down with time to produce the same result. For example, instead of decreasing the current with time according to Equation (1) and Equation (3), through any practical means the average number of coulombs of electricity that traverses through the donor reservoir per dose or per unit time can trend down with time. In this case in Equation (1), I would be the averaged charge flow per dose, and I_{f} would be the charge flow per dose at the final stage of the use of the designed use of the device. For example, to deliver a dose (e.g., 40 μg) initially the current can be turned on for a longer duration per dose, and as time progresses, the dose time is shortened according to Equation (1) and Equation (3).

Further, the fentanyl dose can be delivered with the trending down of charge transferred by a combination of current decrease and dose duration decrease with time. Thus, the trending down of the number of coulombs of electricity that traverses through the donor reservoir per dose or unit time according to Equation (1) and Equation (3) can be effected by a combination of current decrease and dose duration decrease with time. Such systems in which the duration of current flow per dose changes with time is practicable if the system is designed to deliver a dose with a maximum dose periods of equal or less than the dose period at the initial level of delivery. In this way, the dose period will only decrease to result in decreasing charge delivery per unit time, never exceeding the maximum number or duration permissible in the device. With this design, even if at maximum use when the device is activated as much as possible, in such devices with shortened dose periods in the later periods of use the doses will not be delivered immediately one after the other.

The above establishes an exemplary scheme for a current base profile on which the drug delivery current can be controlled to modify to fine-tune the current to result in a more desirable flux profile, e.g., more stable flux profile, such as to approximate a zero-order delivery profile.

### Adjusting Electrotransport Current Based on Conductance or Resistance

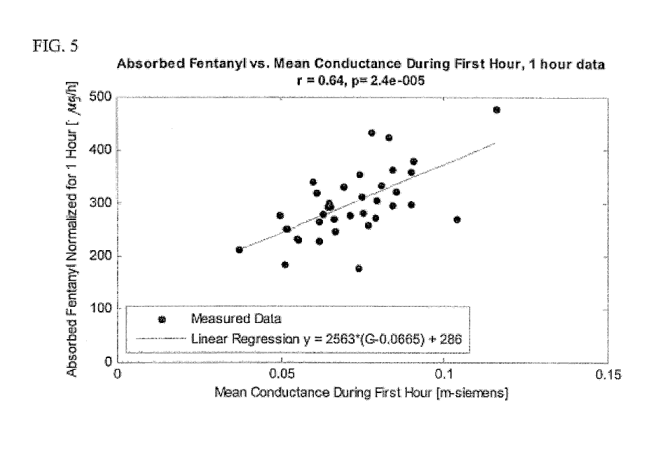

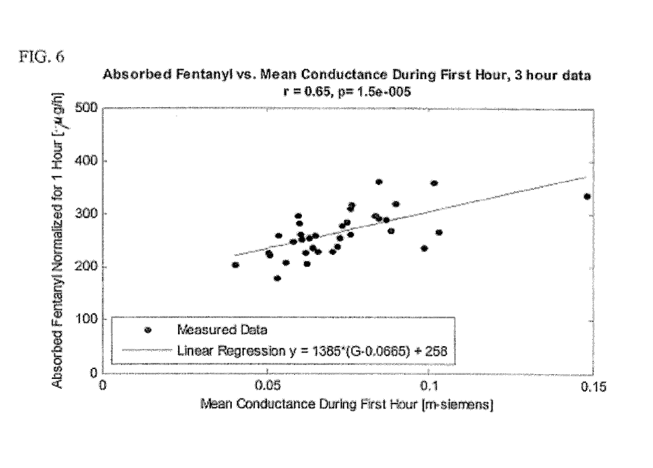

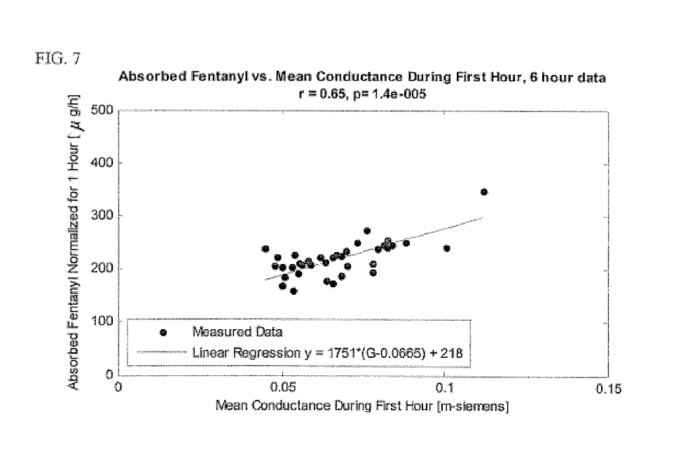

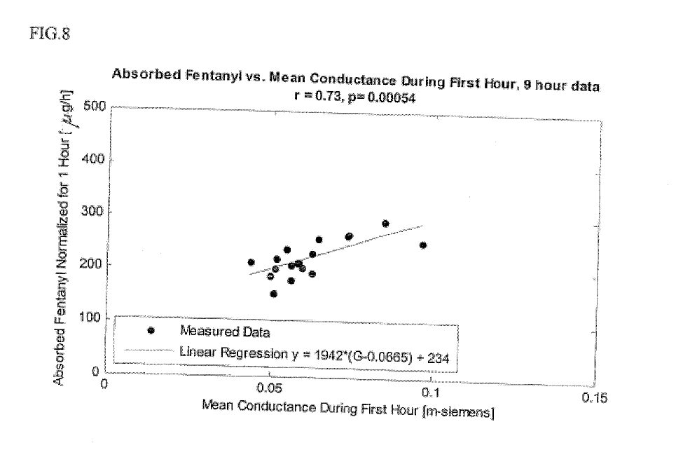

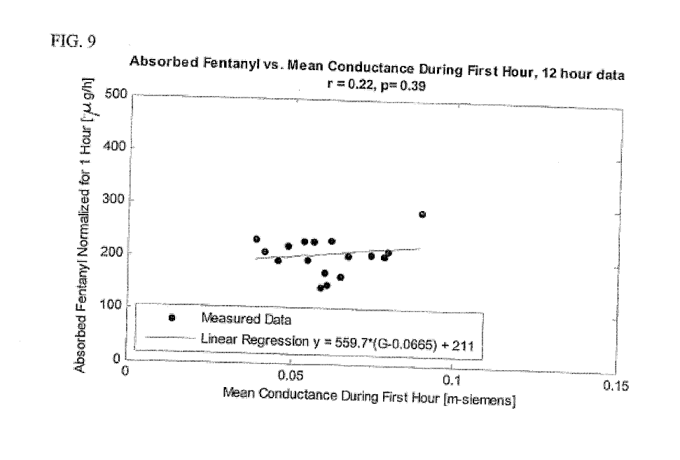

To determine the skin resistance or conductance, skin resistance (and conductance) was calculated from the data of the applied voltage required to drive the current in the experiment associated with Table 7 above on PK1. The applied voltage was measured internally by the device between the donor electrode and the counter electrode. This is one way to sense the resistance or conductance. Thus the resistance would be the resistance between said two electrodes and include the resistance of the donor reservoir, the skin between the reservoirs, and the counter reservoir. The conductance would be the reciprocal of the resistance. For each of the individual tested, for each treatment, the mean output voltage was calculated within each interval. Since the current was constant for each particular set period, the resistance was calculated by dividing the voltage by the current. Similarly, conductance, being the reciprocal of resistance, was calculated. Each dot represents the data for one individual and averaged over that period of time. FIG. 5 is a plot of the average fentanyl absorbed per hour (averaged over the whole treatment) during the entire treatment versus the conductance during the first hour for the individuals in the 1-hour treatment. Conductance is shown instead of resistance because a positive slope is more readily understandable. The ordinate, fentanyl absorbed, represents the total AUC (or total fentanyl absorbed) divided by the length of treatment. For the 1-hour treatment, since drug administration is only for 1 hour, it is the total fentanyl absorbed in the treatment. FIG. 6 is a plot of the average fentanyl absorbed per hour (averaged over the whole treatment) versus the conductance during the first hour for the individuals in the 3-hour treatment. FIG. 7 is a plot of the average fentanyl absorbed per hour (averaged over the whole treatment) versus the conductance during the first hour for the individuals in the 6-hour treatment. FIG. 8 is a plot of the average fentanyl absorbed per hour versus the conductance during the first hour for the individuals in the 9-hour treatment. FIG. 9 is a plot of the average fentanyl absorbed per hour versus the conductance during the first hour for the individuals in the 12-hour treatment. In each graph, the ordinate shows the fentanyl absorbed by the skin from the donor reservoir in units of μg per hour. The abscissa shows the conductance in mSiemens (i.e., 1/Kohm). Since the output voltage to the donor electrode against the return electrode was measured, the output voltage can be assumed to be that delivered to the skin because the resistance of the electrodes and the reservoirs was negligible compared to that of the skin. For example, in FIG. 5 to FIG. 9, the total resistance between the electrodes through the skin was in the order of a few Kohms, whereas we have found that the resistance through a reservoir might be in the order of a few hundred ohms or less. It is noted these graphs are shown as example only. Conductance data from other hours of the tests done for Table 7 were also obtained and showed similar trends in the relation between conductance and flux. Other means of measuring or sensing resistance and conductance using ohmmeters, conductance meters, etc., are known to those skilled in the art and can be implemented as desired.

A linear regression fit was performed for each of the plots of FIG. 5 to FIG. 9. In each of the plots, there is a correlation between the conductance and the amount of fentanyl absorbed per hour, i.e., a correction between conductance and flux. Consequently, there is an inverse correlation between resistance and fentanyl flux, meaning that a higher resistance resulted in a lower flux for the same drug delivery current. It is noted that one skilled in the art can use the resistance (or conductance, or applied voltage in view of current) as a factor for controlling the current or voltage. Further, it is understood that a skilled person can control using such factor values determined between the electrodes (i.e., including the reservoirs and skin), between one electrode and the skin (e.g., measuring only from the donor electrode to the skin contacting the counter reservoir), or just through the skin not including the reservoir. The slope of the fitted curves in FIG. 5 to FIG. 9 generally decreases with treatment length. This shows that the per hour fentanyl absorbance change per conductance difference generally decreases with longer treatment length. In other words, the effect of conductance difference in affecting average fentanyl absorbance decreases with treatment length.

Since the flux goes up with conductance for the same current, to achieve a more consistent flux, preferably the current (and therefore the voltage) is adjusted according to the conductance (or resistance) sensed by the conductance (or resistance) sensor. Thus, if the conductance is high, the current will consequently be reduced, and vice versa. The adjustment can be done, in one embodiment, by determining the difference between the sensed conductance and a predetermined reference point and adjusting the current up or down depending on whether the conductance is below or above the set point by multiplying the difference with a proportionality factor (a proportional slope). FIG. 5 shows a linear regression fit with an equation of w=2563(G−0.0665)+286 wherein w is the fentanyl absorbed averaged for 1 hour (μg/h) and G is the mean conductance in mSiemens. In FIG. 6 (3-hour treatment), the linear regression fit equation is w=1385(G−0.0665)+258. In FIG. 7 (6-hour treatment), the linear regression fit equation is w=1751(G−0.0665)+218. In FIG. 8 (9-hour treatment), the linear regression fit equation is w=1942(G−0.0665)+234. In FIG. 9 (12-hour treatment), the linear regression fit equation is w=559.7(G−0.0665)+211. It is noted that the linear regression fits of FIG. 5 to FIG. 9 show that the slopes (herein referred to as “proportional slope” or “proportional factor”) of the linear regression fit lines vary from about 560 μg/(hr.mSiemen) of the 12 hour data to about 2600 μg/(hr.mSiemen) of the 1 hour data. Data from other hours of the tests done for Table 7 show similar trends of conductance related to flux, that for the same current, fentanyl flux is higher if skin conductance in an individual is higher. Linear regression fit equations for electrotransport drug fentanyl flux versus time for the hours in the study of Table 7 above and FIG. 5 to FIG. 9 show that fentanyl flux is affected by skin conductance and this is true at all hours of the study, which involves different base profile currents. Therefore current adjustment is needed for providing a more consistent flux based on the skin conductance. However, if current is adjusted only to accommodate conductance variation without regard of time, the fact that the amount of flux related to current is not linear but dependent on time has not been taken into consideration and therefore the current adjustment would not be optimal, as data from Studies I, II, and III of the above Table 1 indicate. We have found that the skin conductance goes up within the first two hours of iontophoretic delivery but gradually goes down thereafter (skin resistance will have opposite trend). Although the present invention is not bound by any theory, it is believed that the gradual decrease in conductance after the first two hours is due to the contact of the reservoirs with the skin deteriorating slightly with time and the patient becoming less willing to stay still but moving more with time. The skin conductance time profile differs in its trend somewhat with that of the flux/time profile. Thus, adjusting current using conductance alone without considering time is less optimal than if both time and conductance are taken into account. Therefore, to control fentanyl flux more accurately, the current is preferably adjusted depending on both time and the conductance at that particular time.

Generally, the flux F(t) is proportional to the drug delivery current applied across the reservoirs and the skin. If the current changes with time according to a base profile for achieving a more stable flux as expressed in Equation (3), then F(t) is:

F(t)=m(t)I(t) (4)

In Equation (4), m(t) is a proportional constant that may be a function that changes with time. From the data shown in Study III above, FIG. 4 and Table 5, if desired, m(t) may be approximated by a constant that remains the same, i.e., that the flux is directly proportional to the drug delivery current, i.e., m(t) is a constant. On the other hand, the proportional constant may vary with time, since, for example, the linear fit equations of FIG. 5 to FIG. 9 have different slopes and intersection points with the axes. Thus, it is more desirable that the device is designed to take into account the time variance of m(t). The time variance of m(t) can be determined by experiments similar to those of FIG. 5 to FIG. 9. The data in FIG. 5 to FIG. 9 show that there is a second order effect on flux that can be predicted by the conductance, thus the flux is:

F(t)=m(t)I+n(G−Go) (5)

In Equation (5), I is the drug delivery current according to a base profile (such as that in Equation (3)), n is a proportional factor (i.e., the slope of the flux to conductance relationship, e.g., from graphs such as FIG. 5 to FIG. 9, or an average of the slopes of similar graphs from a population of many individuals), G is the conductance measured and Go is a reference conductance from which the proportional factor is applicable in relation to G. The reference conductance is preferably based on measurements across many individuals as a population average. The slope m(t) that determines the amount of drug delivered for a given current is also a population average. G may be measured at the beginning of a treatment to an individual and used as a one-time adjustment to the current, or it may be measured regularly during delivery.

To keep the flux more stable, it is preferred that the drug delivery current be adjusted to take into account the change in conductance. The applied current can be changed by an amount ΔI based on conductance of an individual to make the flux more predictable from person to person. The flux F(t) in Equation (4) becomes

F(t)=m(t){I+ΔI} (6)

Thus, considering Equations (6) and (5),

m(t){I+ΔI}=m(t)I+n(G−Go). (7)

Therefore, to keep the flux as constant as possible, the change in current based on conductance should be:

$$Δ I = - \frac{n}{m ( t )} ( G - Go ) \qquad ( 8 )$$

Therefore, in Equation (6), I refers to the base profile I(t) according to Equation (2) and ΔI refers to the ΔI according to Equation (8). As a result, to obtain a more stable flux, the drug delivery current preferably varies according to {I+ΔI}, wherein I and ΔI vary as functions of time and conductance according to Equation (3) and Equation (8) respectively.

One way to adjust the current based on the conductance is use the proportional factor to scale up or down the current based on how far the measured conductance is from a reference conductance based on a population average, Go. For delivery of fentanyl, Go ranges from 0.03-0.10 mSiemens, more specifically from 0.5 to 0.75 mSiemens, and in the estimate based on the above-presented studies, it has a value of 0.0665 mSiemens. In one way to adjust the charge used for drug delivery, G was measured as the average conductance measured during the first hour of treatment and Go was calculated based on the average conductance during first hour of treatment for all treatments in all subjects. The conductance measured includes both electrodes, both gels, and the subject's skin. One simpler way to adjust the current for an individual patient is to take a base profile and adjust the base profile using a conductance value obtained in a early period of time during the delivery according to Equation (8). In this case, for the period to time after the early period, G in Equation (8) is a single value, such as the average conductance value during the first hour.

From the data of the above trials, the value of n and m in Equation (8) can be obtained for each period of interest. Table 8 below shows the values of m and n obtained from FIG. 5 to FIG. 9. Consequently the value of n/m in Equation (8) can be calculated from the values of n and m. When the conductance G is measured as the average conductance during the first hour of treatment, the slope n varies from 500-3000 μg/hr/mSiemen, more specifically from 1000-2700 μg/hr/mSiemen. This value varies with elapsed time of treatment and the nominal current delivered. The value of m for fentanyl also varies based on the elapsed time for treatment. It has a range from 0.3 to 1.5 μg/mA, more specifically from 0.5 to 1.25 μg/mA. The value of G will be measured with each application on each individual.

Thus, it is preferred that the adjustment proportional factor (−n/m) based on the sensed conductance is negative and has a magnitude of not less than about 1000 mA/mSiemen or more than about 10,000 mA/mSiemen, preferably about 1000 mA/mSiemen to about 300 mA/mSiemen in magnitude, as calculated from the data plotted in FIG. 6 to FIG. 8, shown in Table 8 below. The equations provided in Table 8 are for one measurement period at the beginning. The conductance was measured once per minute and averaged over the whole first hour. With more frequent measurements, the initial measurement period could be shorter. During each time intervals, the current can be varied from its base amount by an amount ΔI which is calculated from the conductance measured in the first hour, G, using an equation suitable for that period. In this way, an averaged conductance can thus be used for adjusting the current delivery for the whole treatment episode. In Table 8, both n and m change with time. The values of m are derived from Table 7. As illustrated in Table 8, the average conductance for the first hour can be used to adjust the current for the rest of the treatment episode. Similarly, conductance shorter or longer than 1 hour can be similarly averaged and used for adjusting current for the rest of the treatment. If the time period for which averaging is done is different, the equation would then be different from those in Table 8. It is contemplated that the amount of drug delivered will be more precise with more regular measurement and update.

TABLE 8

| | m | n | −n/m | Change in current |
|---|---|---|---|---|
| Interval | (μg/mA/hr) | (μg/mSiemen/hr) | (mA/(mSiemen)) | (mA), G (mSiemens) |
| 0-1 | 0.79 | 2563 | −3244 | ΔI = −3244(G-0.0665) |
| hour | | | | |
| 1-3 | 0.83 | 1385 | −1669 | ΔI = −1669(G-0.0665) |
| hours | | | | |
| 3-6 | 0.76 | 1751 | −2172 | ΔI = −2172(G-0.0665) |
| hours | | | | |
| 6-9 | 1.21 | 1942 | −1605 | ΔI = −1605(G-0.0665) |
| hours | | | | |

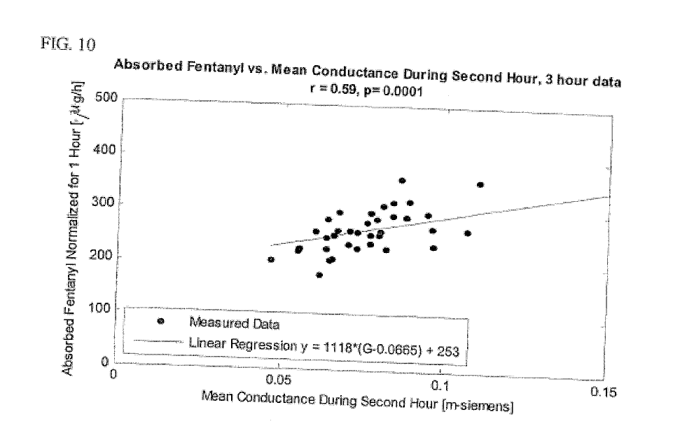

As an alternative of using the conductance for the first hour as G, more than one average conductance can be used for adjusting the current delivery for the whole treatment. For example, the value of G can be measured for each separate period and be used for adjusting the current for that period or for a subsequent period based on the equations above. For example, FIG. 10 shows a plot of the fentanyl absorbed versus the conductance during the second hour for the individuals in the 3-hour treatment obtained from the data of Study III. As a comparison, the curve fitting of FIG. 10 can be used in a similar way as the first hour plot for the 3-hour treatment of FIG. 6. Similarly, plots and curve fitting equations can be obtained from the second hour for the 6-hour treatment, the 9-hour treatment, and the 12-hour treatments corresponding to those of FIG. 7 to FIG. 9. Further, the conductance versus fentanyl absorbance for the other hours can be obtained and used for the current adjustment. For a more refined current adjustment with conductance and time, the conductance for each period or hour can be measured and used for adjusting the current based on the values of n and m previously calculated from curve fitting from data such as those in Study III.

The factors m and n can be applied for averaged fentanyl absorption (normalized for 1 hour) of 100 to 400 μg/hr, preferably 200 to 300 μg/hr. For the proportional factors to estimate current adjustment needed to maintain a more stable flux, a reference conductance point can be chosen by various means for example, by taking the average conductance at a particular flux of the individuals of one or more flux-conductance plots like FIG. 5 to FIG. 9 (if using the first hour conductance G as a basis for adjustment) or selecting a point from a plot that gives the desired flux (e.g., a data point on a linear regression fit line). For example, the reference conductance point can be chosen as a point on the linear regression fit line at a particular fentanyl flux (e.g., at the fentanyl flux of 200 μg/hr on the linear regression line on FIG. 7). Also, more clinical experiments can be done to generate more plots to obtain data points that approach the desired flux (e.g., that which would actually provide 40 μg of drug in a 10 min dose period, which is equivalent to a flux of 240 μg/hr, for a nominal 40 μg per dose akin to IONSYS that delivers a dose over 10 min). Optionally, the controller can include a limit so the current will not be adjusted to exceed a certain limit. The controller can also include an algorithm to calculate the fentanyl absorption rate resulting from the adjusted current based on the linear regression fit model so as to determine whether the calculated fentanyl absorption rate has approached the target rate and stop further adjusting the current past the target rate.

Other than simply using a proportional factor based on a linear model, one skilled in the art will understand that the adjustment can be done with a model that is not exactly proportional with respect to a numerical proportional factor, so long as the current (or rate of charge flow) is adjusted upward with a low conductance and adjusted downward with a high conductance. For example, the adjustment, instead of basing on a constant slope (relating to a proportional factor) of a linear regression fit, can be based on a curve that has a general trend of raising the current with a low sensed conductance and reducing the current with a high sensed conductance.

Further, the adjustment algorithm (i.e., the programming of the controller in the device) can be set in such a way that the adjustment preferably depends on both the conductance and the time during the use of the device. For example, in a base profile of drug delivery current similar to that of Table 6 in which different periods have different average current levels, different conductance dependency can be used for different times, e.g., for each different period. As FIG. 5 to FIG. 9 show, the proportional slopes or proportional factors are different from period to period. Such adjustments can be done with discrete steps in the current base profile and the proportional factors. Of course, one skilled in art, based on the present invention disclosure, will be able to device a continually changing, smoother adjustment scheme with less dramatic transitions. Therefore, preferably, in the device the drug delivery current trends down with time generally, and further that the drug delivery current is controlled to depend inversely on the conductance, in that high conductance leads to the reduction of current flow by the controller.

Even if the conductance (or reciprocally resistance) changes with time, it is preferred that the current be adjusted at a given moment not only on the conductance (or reciprocally resistance) sensed, which is already time dependent in such cases, but also depend on the time such conductance-sensing and current-adjustment is made. Thus, in a preferred mode, adjusting the current based on time and based on conductance is different from adjusting the current based on a time dependent conductance alone without additionally taking the time of the conductance value separately as a factor.

The present invention is especially useful for the device to be suitable for a broad spectrum of individuals. Because people are different physiologically, different individuals would have different skin conductance and resistance. Thus, a device of the present invention can automatically adjust the current based on the conductance or resistance it senses, thereby providing the desired amount of fentanyl flux.

It is understood that the sensing of resistance is equivalent to sensing conductance. Further, sensing voltage or current while knowing the other is equivalent to sensing conductance or resistance because resistance and conductance can be calculated from voltage and current data. The controller, or other circuitry can easily be programmed or set to use voltage and current data to achieve the goal of adjusting the current and/or voltage to provide a fentanyl flux that is near to what the device is designed to deliver (such as 40 μg/dose according to regulatory approval by the drug approval agency of a government) and provide a more consistent and stable flux over time.

Although a useful embodiment of the present invention is one in which a DC (direct current) of a constant amplitude is applied for a period of time that is minutes long (e.g., 30 min, 60 min, 180 min, etc.) for each of the stepped down current levels, devices in which the current is applied as pulsed current of constant amplitude for each of the periods with stepped down amplitude can also be used. Typically the pulses have pulse width in terms of milliseconds to tens of seconds. The pulse amplitude for all the cycles in the “on” phase is the same during a period of a particular step in the stepped down delivery. When the device is at a particular step in the stepped down delivery, the current at a constant level in the “on” phase of a duty cycle can remain the same during that particular step. For adjusting the current in response to conductance changes, the amplitude, the length of the duty cycle, and the length of the “on” phase can be controlled to provide the charge delivery or current profile desired.

Safeguard measures can be implemented, for example, in setting limits for any particular period of drug delivery so as to prevent from overdosing or underdosing. Such safeguard measures can be done by setting limits to the total charge delivered for each individual period and the limits can be different for different periods.

It is noted that although it is desirable to control the current delivery in a dose period to result in the amount of charge as a function of both time and resistance (or its inverse, conductance), if desired, the present invention can be practiced in a modified respect by controlling the current delivery so that the amount of charge delivered in a dose period is only a function of the resistance (or its inverse, conductance). A person skilled in the art will be able to implement such a scheme in view of the present disclosure by utilizing, e.g., Equation (6), i.e., F(t)=m(t) {I+ΔI}, by keeping the base profile of current I constant instead of varying the current base profile I according to time.

The above-described exemplary embodiments are intended to be illustrative in all respects, rather than restrictive, of the present invention. Thus the present invention is capable of many variations in detailed implementation that can be derived from the description contained herein by a person skilled in the art, e.g., by permutation or combination of various features. Although specific iontophoretic devices are described in detail as illustration, other modifications are possible by one skilled in the art. All such variations and modifications are considered to be within the scope of the present invention. The entire disclosure of each patent, patent application, and publication cited or described in this document is hereby incorporated herein by reference.

What is claimed is:

1. A transdermal electrotransport system for administering fentanyl through the skin of a user, comprising:
(i) donor reservoir comprising fentanyl salt;
(ii) at least two electrodes for conducting a current to drive fentanyl ions from the donor reservoir transdermally to deliver a dose of fentanyl in a dose period;
(iii) sensor for sensing a factor that reflects one of resistance of the skin to current and conductance of the skin to current;
(iv) a controller for controlling current delivery that drives the fentanyl ions such that the amount of charge per dose period of the current passing through the donor reservoir varies as a function of time and the factor to result in a more stable fentanyl delivery than a constant charge per dose period delivery.

2. The system of claim 1 wherein the controller controls the current delivery such that the amount of charge per dose period passing through the donor reservoir decreases with time nonlinearly and nonexponentially and at a particular time, increasing the current delivery if a higher resistance or a lower conductance is sensed and decreasing the current delivery if a lower resistance or a higher conductance is sensed.

3. The system of claim 2 wherein the controller controls the current delivery to have a time varying base profile and adjusts the current delivery by increasing or decreasing the current delivery from the base profile according to the resistance or conductance sensed.

4. The system of claim 3 wherein the controller adjusts the current delivery to vary from the base profile with a function that is time dependent and dependent on the resistance or conductance sensed.

5. The system of claim 3 wherein the controller adjusts the current delivery to vary from the base profile with a function that is time dependent and according to the resistance or conductance sensed, wherein the adjustment from the base profile is negative in with a magnitude less than 10,000 mA/mSiemen.

6. The system of claim 3 wherein the sensor senses the resistance or conductance by sending a test current to the skin.

7. The system of claim 3 wherein the controller controls the current delivery such that according to the base profile an average amount of charge per dose period trends down for the first 6 hours.

8. The system of claim 2 wherein the controller controls the device to deliver from 30 μg to 60 μg of fentanyl per dose.

9. The system of claim 3 wherein an average conductance for a period is used to adjust the current delivery for a whole episode of treatment.

10. The system of claim 3 wherein more than one time averaged conductance is used to adjust the current delivery for a whole episode of treatment.

11. The system of claim 3 wherein the controller controls the current delivery to discrete periods of constant current delivery for at least a period of fentanyl delivery.

12. The system of claim 4 wherein the controller limits how much the current delivery can be adjusted so that the current delivery does not exceed a maximum or fall below a minimum.

13. The system of claim 3 wherein the controller controls the device to deliver from 35 μg to 50 μg of fentanyl per dose.

14. The system of claim 3 wherein the donor reservoir has a surface area for contacting skin of 0.8 cm² to 10 cm².

15. The system of claim 3 wherein the donor reservoir comprises fentanyl halide.

16. The system of claim 3 wherein the donor reservoir comprises fentanyl hydrochloride.

17. The system of claim 3 wherein the controller controls the current delivery to substantially continually change with time for at least a period of fentanyl delivery.

18. A method for transdermal electrotransport of fentanyl through the skin, comprising:
(i) sensing a factor that reflects one of resistance of the skin to current and conductance of the skin to current;
(ii) controlling current delivery to a fentanyl reservoir to drive fentanyl ions therefrom through the skin such that the amount of charge per dose period passing through the donor reservoir varies as a function of time and the factor to result in a more stable fentanyl delivery than a constant charge per dose period delivery.

19. The method of claim 18, comprising controlling the current delivery such that the amount of charge per dose period passing through the donor reservoir decreases with time nonlinearly and nonexponentially and at a particular time, increasing the current delivery if a higher resistance or a lower conductance is sensed and decreasing the current delivery if a lower resistance or a higher conductance is sensed.

20. The method of claim 19, comprising controlling the current delivery to have a time varying base profile and adjusting the current delivery by increasing or decreasing the current delivery from the base profile according to the resistance or conductance sensed.

21. The method of claim 19, comprising adjusting the current delivery to vary from the base profile with a function that is time dependent and according to the resistance or conductance sensed, wherein the adjustment from the base profile is negative in with a magnitude less than 10,000 mA/mSiemen.

22. The method of claim 19, comprising sensing the resistance or conductance by sending a test current to the skin.

23. The method of claim 19, comprising controlling the current delivery such that an average amount of charge per dose period averaged over an earlier hourly period does not differ from an average amount of charge per dose period of an immediately subsequent hourly period by more than 25% of the average current delivery of the earlier hourly periods.

24. The method of claim 19, comprising controlling the current delivery to deliver from 30 μg to 60 μg of fentanyl per dose.

25. The method of claim 19, comprising controlling the current delivery such that the current delivery trends down for the first 6 hours.

26. The method of claim 19, comprising using an average conductance for a period is to adjust the current delivery for a whole episode of treatment.

27. The method of claim 19, comprising using more than one time averaged conductance to adjust the current delivery for a whole episode of treatment.

28. The method of claim 19, comprising controlling the current delivery to deliver from 35 μg to 50 μg of fentanyl per dose.

29. The method of claim 19, comprising delivering fentany through a donor reservoir having a surface area for contacting skin of 0.8 cm² to 10 cm².

30. The method of 19, comprising delivering fentany from a donor reservoir that comprises fentanyl halide.

31. A method for making a transdermal electrotransport system for administering fentanyl through the skin, comprising:
(i) providing at least two electrodes, one of which for conducting a drug delivery current to drive fentanyl ions from a fentanyl reservoir having fentanyl salt;
(ii) providing a sensor for sensing a factor that reflects one of resistance of the skin to current and conductance of the skin to current;
(iii) connecting a controller to the at least two electrodes for controlling the drug delivery current that drives the fentanyl ions to deliver a dose of fentanyl in a dose period such the average amount of charge passing through the donor reservoir per dose period passing through the donor reservoir varies as a function of time and the factor and decreases with time nonlinearly and nonexponentially for doses at different time periods to improve stability of fentanyl delivery than a constant charge per dose period delivery.
